SEQUENCE LISTING (81 sequences)

SEQ ID NO: 1 - DNA, 1243 bp, Homo sapiens
ccatgggcttcccaaccattcccttatccaggctttttgacaacgctagtctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggccgcggtggcggaggtagtggtggcggaggtagcggtggcggaggttctggtggcggaggttccgaattcttcccaaccattcccttatccaggctttttgacaacgctagtctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcaagcttttcgaata

SEQ ID NO: 2 - protein, 413 aa, Homo sapiens
MGFPTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGRGGGGSGGGGSGGGGSGGGGSEFFPTIPLSRLFDNASLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFKLFE

SEQ ID NO: 3 - protein, 7 aa, artificial sequence
  helical peptide
AEAAAKA

SEQ ID NO: 4 - protein, 12 aa, artificial sequence
  two copies of helical peptide
AEAAAKEAAAKA

SEQ ID NO: 5 - protein, 17 aa, artificial sequence
  three copies of helical peptide
AEAAAKEAAAKEAAAKA

SEQ ID NO: 6 - DNA, 28 bp, artificial sequence
  primer encoding helical peptide
ggccgcgcagaggctgcggccaaggcag

SEQ ID NO: 7 - DNA, 28 bp, artificial sequence
  primer encoding helical peptide
aattctgccttggccgcagcctctgcgc

SEQ ID NO: 8 - DNA, 43 bp, artificial sequence
  primer encoding helical peptide
ggccgcgcagaggccgcagctaaagaagctgcggccaaggcag

SEQ ID NO: 9 - DNA, 43 bp, artificial sequence
  primer encoding helical peptide
aattctgccttggccgcagcttctttagctgcggcctctgcgc

SEQ ID NO: 10 - DNA, 58 bp, artificial sequence
  primer 6
ggccgcgcagaggccgcagctaaagaagcggcagccaaggaagctgcggccaaggcag

SEQ ID NO: 11 - DNA, 58 bp, artificial sequence
  primer encoding helical peptide
aattctgccttggccgcagcttccttggctgccgcttctttagctgcggcctctgcgc

SEQ ID NO: 12 - DNA, 42 bp, artificial sequence
  primer encoding helical peptide
ggccgcgcagaggccgcagctaaagaagcggcagccaaggag

SEQ ID NO: 13 - DNA, 31 bp, artificial sequence
  primer encoding helical peptide
gcagccgctaaagaagctgcggccaaggcag

SEQ ID NO: 14 - DNA, 42 bp, artificial sequence
  primer encoding helical peptide
aattctgccttggccgcagcttctttagcggctgcctccttg

SEQ ID NO: 15 - DNA, 31 bp, artificial sequence
  primer encoding helical peptide
gctgccgcttctttagctgcggcctctgcgc

SEQ ID NO: 16 - protein, 16 aa, artificial sequence
  linker to link GH molecules
QARAEAAAAAAKASRL

SEQ ID NO: 17 - DNA, 48 bp, artificial sequence
  nucleic acid encoding linker amino acid sequence
caagcacgagcagaagcagcagcagcagcagcaaaagcatcacgacta

SEQ ID NO: 18 - DNA, 48 bp, artificial sequence
  nucleic acid sequence encoding linker sequence
caggctcgtgctgaggctgctgctgctgctgctaaggcttctcgtctt

SEQ ID NO: 19 - DNA, 48 bp, artificial sequence
  nucleic acid sequence encoding linker sequence
caagcccgcgccgaagccgccgccgccgccgccaaagcctcccgcctc

SEQ ID NO: 20 - DNA, 48 bp, artificial sequence
  nucleic acid sequenc encoding linker sequence
caagcgcgggcggaagcggcggcggcggcggcgaaagcgtcgcggctg

SEQ ID NO: 21 - DNA, 48 bp, artificial sequence
  nucleic acid sequence encoding linker sequence
caagcaagagcagaagcagcagcagcagcagcaaaagcatcaagatta

SEQ ID NO: 22 - DNA, 48 bp, artificial sequence
  nucleic acid sequence encoding linker sequence
caagcaagggcagaagcagcagcagcagcagcaaaagcatcaaggttg

SEQ ID NO: 23 - DNA, 8 bp, artificial sequence
  restriction site for Not 1
gcggccgc

SEQ ID NO: 24 - DNA, 6 bp, artificial sequence
  restriction site for Nru I
tcgcga

SEQ ID NO: 25 - DNA, 6 bp, artificial sequence
  restriction site for SacII
ccgcgg

SEQ ID NO: 26 - DNA, 32 bp, artificial sequence
  primer for modifying GH1 and GH2 domains
aaaccatgggcttcccaaccattcccttatcc

SEQ ID NO: 27 - DNA, 43 bp, artificial sequence
  primer for modifying GH1 and GH2 domains
agtagtagtagagcggccgcctctgcgcgggcctgcacgatgc

SEQ ID NO: 28 - DNA, 38 bp, artificial sequence
  primer for modifying GH1 and GH2 domains
ttccgaattctcgcgactttttgacaacgctagtctcc

SEQ ID NO: 29 - DNA, 29 bp, artificial sequence
  primer for modifying GH1 and GH2 domains
agccaagcttgaagccacagctgccctcc

SEQ ID NO: 30 - DNA, 102 bp, artificial sequence
  tandem linker domain
caggcccgcgcagaggcggccgcggtggcggaggtagtggtggcggaggtagcggtggcggaggttctggtggcggaggttccgaattctcgcgactttttg

SEQ ID NO: 31 - protein, 35 aa, artificial sequence
  tandem linker domain amino acid sequence
QARAEAAAVAEVVVAEVAVAEVLVAEVPNSRDFLL

SEQ ID NO: 32 - DNA, 22 bp, artificial sequence
  nucleic acid sequence used to generate rigid helical linker
ggccgctaaagccgcggcttcg

SEQ ID NO: 33 - DNA, 18 bp, artificial sequence
  primer used to generate rigid helical linker
cgaagccgcggctttagc

SEQ ID NO: 34 - DNA, 31 bp, artificial sequence
  primer used to generate rigid helical linker
ggccgctaaagaagccgcggctaaggcatcg

SEQ ID NO: 35 - DNA, 27 bp, artificial sequence
  primer used to generate rigid helical linker
cgatgccttagccgcggcttctttagc

SEQ ID NO: 36 - DNA, 34 bp, artificial sequence
  primer used to generate rigid helical linker
ggccgctaaagaagccgcggctaaggcagcttcg

SEQ ID NO: 37 - DNA, 30 bp, artificial sequence
  primer used to generate rigid helical linker
cgaagctgccttagccgcggcttctttagc

SEQ ID NO: 38 - DNA, 37 bp, artificial sequence
  primer used to generate rigid helical linker
ggccgctaaagaagccgcggctaaggcagctgcgtcg

SEQ ID NO: 39 - DNA, 33 bp, artificial sequence
  primer used to generate rigid helical linker
cgacgcagctgccttagccgcggcttctttagc

SEQ ID NO: 40 - DNA, 40 bp, artificial sequence
  primer used to generate rigid helical linker
ggccgctaaagaagccgcggctaaggcagctgcggcatcg

SEQ ID NO: 41 - DNA, 36 bp, artificial sequence
  primer used to generate rigid helical linker
cgatgccgcagctgccttagccgcggcttctttagc

SEQ ID NO: 42 - DNA, 46 bp, artificial sequence
  primer used to generate rigid helical linker
ggccgctaaagaagccgcggctaaggcagaggctgcggccaaatcg

SEQ ID NO: 43 - DNA, 42 bp, artificial sequence
  primer used to generate rigid helical linker
cgatttggccgcagcctctgccttagccgcggcttctttagc

SEQ ID NO: 44 - DNA, 49 bp, artificial sequence
  primer used to generate rigid helical linker
ggccgctaaagaagccgcggctaaggcagaggctgcggccaaagcttcg

SEQ ID NO: 45 - DNA, 45 bp, artificial sequence
  primer used to generate rigid helical linker
cgaagctttggccgcagcctctgccttagccgcggcttctttagc

SEQ ID NO: 46 - DNA, 52 bp, artificial sequence
  primer used to generate rigid helical linker
ggccgctaaagaagccgcggctaaggcagaggctgcggccaaagctgcgtcg

SEQ ID NO: 47 - DNA, 48 bp, artificial sequence
  primer used to generate rigid helical linker
cgacgcagctttggccgcagcctctgccttagccgcggcttctttagc

SEQ ID NO: 48 - DNA, 2016 bp, artificial sequence
  nucleic acid encoding growth hormone fused to growth hormone receptor extracellular domain
ttcccaaccattcccttatccaggctttttgacaacgctagtctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcctcgagggtggcggaggtagtggaggcggtggctctggcggtggaggctccggaggcggtggatcaaaggatccaacccttttcccaaccattcccttatccaggctttttgacaacgctagtctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggccgcggtggcggaggtagtggtggcggaggtagcggtggcggaggttctggtggcggaggttccgaattcttttctggaagtgaggccacagcagctatccttagcagagcaccctggagtctgcaaagtgttaatccaggcctaaagacaaattcttctaaggagcctaaattcaccaagtgccgttcacctgagcgagagactttttcatgccactggacagatgaggttcaccatggaacaaagaacctaggacccatacagctgttctataccagaaggaacactcaagaatggactcaagaatggaaagaatgccctgattatgtttctgctggggaaaacagctgttactttaattcatcgtttacctccatctggataccttattgtatcaagctaactagcaatggtggtacagtggatgaaaagtgtttctctgttgatgaaatagtgcaaccagatccacccattgccctcaactggactttactgaacgtcagtttaactgggattcatgcagatatccaagtgagatgggaagcaccacgcaatgcagatattcagaaaggatggatggttctggagtatgaacttcaatacaaagaagtaaatgaaactaaatggaaaatgatggaccctatattgacaacatcagttccagtgtactcattgaaagtggataaggaatatgaagtgcgtgtgagatccaaacaacgaaactctggaaattatggcgagttcagtgaggtgctctatgtaacacttcctcagatgagccaa

SEQ ID NO: 49 - protein, 672 aa, artificial sequence [sequence omitted]

SEQ ID NO: 50 - DNA, 1217 bp, artificial sequence
  growth hormone tandem fused via semi-rigid linker
ccatgggcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcggcggccgcgaattcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcaagcttttcgaataaatcgatgtcgagcaccaccaccaccaccactga

SEQ ID NO: 51 - protein, 403 aa, artificial sequence
  growth hormone tandem fused via semi-rigid linker
MGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFGGREFFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFKLFEIDVEHHHHHH

SEQ ID NO: 52 - DNA, 36 bp, artificial sequence
  nucleic acid sequence encoding semi-rigid linker
gcagaggccgcagctaaagaagctgcggccaaggca

SEQ ID NO: 53 - protein, 12 aa, artificial sequence
  semi-rigid linker amino acid sequence
AEAAAKEAAAKA

SEQ ID NO: 54 - DNA, 66 bp, artificial sequence
  nucleic acid sequence encoding semi-rigid linker
gcagaggccgcagctaaagaagcggcagccaaggaggcagccgctaaagaagctgcggccaaggca

SEQ ID NO: 55 - protein, 22 aa, artificial sequence
  semi-rigid linker amini acid sequence
AEAAAKEAAAKEAAAKEAAAKA

SEQ ID NO: 56 - DNA, 1174 bp, artificial sequence
  growth hormone tandem linked via a rigid linker
ccatgggcttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcaggcccgcgcagaggcggccgctcgcgactttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttcaagcttttcgaataaatcgatgtcgagcaccaccaccaccaccactga

SEQ ID NO: 57 - protein, 389 aa, artificial sequence
  amino acid sequence growth hormone tandem linked via rigid linker
MGFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQARAEAAASRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGFKLFEIDVEHHHHHH

SEQ ID NO: 58 - protein, 7 aa, artificial sequence
  rigid linker
KEAAAKA

SEQ ID NO: 59 - protein, 8 aa, artificial sequence
  rigid linker 2
KEAAAKAA

SEQ ID NO: 60 - protein, 9 aa, artifical sequence
KEAAAKAAA

SEQ ID NO: 61 - protein, 10 aa, artificial sequence
  rigid linker
KEAAAKAAAA

SEQ ID NO: 62 - protein, 12 aa, artificial sequence
  rigid linker
KEAAAKAEAAAK

SEQ ID NO: 63 - protein, 13 aa, artificial sequence
  rigid linker
KEAAAKAEAAAKA

SEQ ID NO: 64 - protein, 14 aa, artificial sequence
  rigid linker
KEAAAKAEAAAKAA

SEQ ID NO: 65 - DNA, 597 bp, Homo sapiens
ttgcccatctgtcccggcggggctgcccgatgccaggtgacccttcgagacctgtttgaccgcgccgtcgtcctgtcccactacatccataacctctcctcagaaatgttcagcgaattcgataaacggtatacccatggccgggggttcattaccaaggccatcaacagctgccacacttcttcccttgccacccccgaagacaaggagcaagcccaacagatgaatcaaaaagactttctgagcctgatagtcagcatattgcgatcctggaatgagcctctgtatcatctggtcacggaagtacgtggtatgcaagaagccccggaggctatcctatccaaagctgtagagattgaggagcaaaccaaacggcttctagagggcatggagctgatagtcagccaggttcatcctgaaaccaaagaaaatgagatctaccctgtctggtcgggacttccatccctgcagatggctgatgaagagtctcgcctttctgcttattataacctgctccactgcctacgcagggattcacataaaatcgacaattatctcaagctcctgaagtgccgaatcatccacaacaacaactgc

SEQ ID NO: 66 - protein, 199 aa, Homo sapiens
LPICPGGAARCQVTLRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLEGMELIVSQVHPETKENEIYPVWSGLPSLQMADEESRLSAYYNLLHCLRRDSHKIDNYLKLLKCRIIHNNNC

SEQ ID NO: 67 - DNA, 555 bp, Homo sapiens
cttcgagacctgtttgaccgcgccgtcgtcctgtcccactacatccataacctctcctcagaaatgttcagcgaattcgataaacggtatacccatggccgggggttcattaccaaggccatcaacagctgccacacttcttcccttgccacccccgaagacaaggagcaagcccaacagatgaatcaaaaagactttctgagcctgatagtcagcatattgcgatcctggaatgagcctctgtatcatctggtcacggaagtacgtggtatgcaagaagccccggaggctatcctatccaaagctgtagagattgaggagcaaaccaaacggcttctagagcgcatggagctgatagtcagccaggttcatcctgaaaccaaagaaaatgagatctaccctgtctggtcgggacttccatccctgcagatggctgatgaagagtctcgcctttctgcttattataacctgctccactgcctacgcagggattcacataaaatcgacaattatctcaagctcctgaagtgccgaatcatccacaacaacaactgc

SEQ ID NO: 68 - protein, 185 aa, Homo sapiens
LRDLFDRAVVLSHYIHNLSSEMFSEFDKRYTHGRGFITKAINSCHTSSLATPEDKEQAQQMNQKDFLSLIVSILRSWNEPLYHLVTEVRGMQEAPEAILSKAVEIEEQTKRLLERMELIVSQVHPETKENEIYPVWSGLPSLQMADEESRLSAYYNLLHCLRRDSHKIDNYLKLLKCRIIHNNNC

SEQ ID NO: 69 - DNA, 573 bp, Homo sapiens
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttc

SEQ ID NO: 70 - protein, 191 aa, Homo sapiens [sequence omitted]

SEQ ID NO: 71 - DNA, 573 bp, Homo sapiens
ttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaacgcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttc

SEQ ID NO: 72 - protein, 191 aa, Homo sapiens [sequence omitted]

SEQ ID NO: 73 - DNA, 8 bp, artificial sequence
  Restriction site for Nco 1
ccatgggc

SEQ ID NO: 74 - DNA, 6 bp, artificial sequence
  restriction site for EcoR1
gaattc

SEQ ID NO: 75 - DNA, 6 bp, artificial sequence
  restriction site for HindIII
aagctt

SEQ ID NO: 76 - DNA, 35 bp, artificial sequence
  GH linker domain
caggcccgcgcagaggcggccgcgtcgcgactttt

SEQ ID NO: 77 - protein, 11 aa, artificial sequence
  GH rigid linker domain
QARAEAAASRL

SEQ ID NO: 78 - DNA, 35 bp, artificial sequence
  nucleic acid sequence encoding linker between growth hormone and prolactin
ctcctgaaggcagaggcggccgcgtcgcgactttt

SEQ ID NO: 79 - protein, 11 aa, artificial sequence
  linker domain linking growth hormone with prolactin
LLKAEAAASRL

SEQ ID NO: 80 - DNA, 33 bp, artificial sequence
  nucleic acid sequence encoding linker domain linking prolactin to prolactin
ctcctgaaggcagaggcggccgcgcttcgagac

SEQ ID NO: 81 - protein, 11 aa, artificial sequence
  linker domain linking prolactin to prolactin
LLKAEAAALRD

(19) United States
(12) Patent Application Publication
(10) Pub. No.: US 2009/0221477 A1
(43) Pub. Date: Sep. 3, 2009

(54) Linkers

(75) Inventors: Peter Artymiuk, Sheffield (GB); Sarbendra Pradhananga, Sheffield (GB); Jon Sayers, Sheffield (GB); Richard Ross, Sheffield (GB)

Correspondence Address: CROWELL & MORING LLP;INTELLECTUAL PROPERTY GROUP, P.O. BOX 14300, WASHINGTON, DC, 20044-4300, US

(73) Assignee: Asterion Limited, Sheffield (GB)

(21) Appl. No.: 11/658,526

(22) PCT Filed: Jul. 18, 2005
(86) PCT No.: PCT/GB2005/002826

§ 371 Date: Apr. 16, 2009

Related U.S. Application Data
(60) Provisional application No. 60/591,358, filed on Jul. 26, 2004.

(30) Foreign Application Priority Data
Jul. 27, 2004 (GB) 0416687.2
Feb. 11, 2005 (GB) 0502839.4

(51) Int. Cl. A61K 38/16; C07K 14/00; C07H 21/04; C12N 5/10; C12N 1/00; C12P 21/00; A61P 37/00

(52) U.S. Cl. 514/12; 530/350; 536/23.1; 435/325; 435/243; 435/69.1

(57) ABSTRACT

We disclose therapeutic polypeptides comprising at least two domains capable of binding to a cytokine receptor, wherein the domains are connected by a peptide linker, wherein the linker optionally comprises a rigid alpha helical region.

The invention relates to polypeptides comprising at least two domains capable of binding to a cytokine receptor, wherein the domains are connected by a peptide linker molecule.

A group of growth factors, referred to as cytokines, are involved in a number of diverse cellular functions. These include, by example and not by way of limitation, modulation of the immune system, regulation of energy metabolism and control of growth and development. Cytokines mediate their effects via receptors expressed at the cell surface on target cells. Cytokine receptors can be divided into three separate sub groups. Type 1 (growth hormone (GH) family) receptors are characterised by four conserved cysteine residues in the amino terminal part of their extracellular domain and the presence of a conserved Trp-Ser-Xaa-Trp-Ser motif in the C-terminal part. The repeated Cys motif are also present in Type 2 (interferon family) and Type III (tumour necrosis factor family).

It is known that many cytokine domains interact with their cognate receptor via specific sites. Some cytokine receptors have both high affinity domain binding sites and low affinity binding sites.

For example, it is known that a single molecule of GH associates with two receptor molecules (GHR) (Cunningham et al., 1991; de Vos et al., 1992; Sundstrom et al., 1996; Clackson et al., 1998). This occurs through two unique receptor-binding sites on GH and a common binding pocket on the extracellular domain of two receptors. Site 1 on the GH molecule has a higher affinity than site 2, and receptor dimerization is thought to occur sequentially with one receptor binding to site 1 on GH followed by recruitment of a second receptor to site 2. The extracellular domain of the GHR exists as two linked domains each of approximately 100 amino acids. It is a conformational change in these two domains that occurs on hormone binding with the formation of the trimeric complex GHR-GH-GHR. Internalisation of the GHR-GH-GHR complex is followed by a recycling step whereby the receptor molecule is regenerated for further use within the cell.

Cytokines and other domains often form receptor-domain complexes upon binding. The receptors involved in the complex formation may be homogenous or heterogeneous. For example, erythropoietin and GH, form a trimeric receptor-hormone-receptor complex. Interleukin-4 forms a trimeric receptor-hormone-different receptor complex. Tumour necrosis factor signals via the formation of homotypic trimers of the cell transmembrane tumour necrosis factor receptors; TNF-1/p55 or TNF-2/p75. Other cytokines, for example leptin and GCSF, form tetrameric receptor-hormone-hormone-receptor complexes, and others (e.g. interleukin 6) probably form hexameric complexes consisting of two soluble receptor molecules, two transmembrane receptor molecules and two cytokine molecules. In each case there is a primary high affinity binding site that locates the cytokine to the receptor complex, and additional sites that play secondary roles in altering the conformation or recruiting other molecules and thereby initiating signalling.

The TNF super-family of cytokines activate signalling pathways for cell survival, death and differentiation that regulate the development, organization and homeostasis of lymphoid, mammary, neuronal and ectodermal tissues. TNF has a demonstrated role in host defense, such roles include for example, splenic cell differentiation, complete IgG response and isotype switching, activation of macrophages, generation of nitric oxide and reactive oxygen radicals. However, TNF is also involved in pathogenesis when over-expressed. Evidence for such an involvement has be found in the following pathologies; bacterial sepsis; graft-versus-host disease; cerebral malaria; rheumatoid arthritis; alopecia areata/generalis; asthma; cancer; Crohn's disease; diabetes; obesity; psoriasis and psoriatic arthritis; sarcoidosis; scleroderma and toxic shock syndrome. These pathologies are recognised as established and/or potential pathologies for applications of anti-TNF agents.

The over-expression of cytokines is the cause of a range of human diseases, for example acromegaly; gigantism; GH deficiency; Turners Syndrome; renal failure; osteoporosis; osteoarthritis; diabetes mellitus; cancer; obesity; insulin resistance; hyperlipidaemia; hypertension; anaemia; autoimmune and infectious disease; inflammatory disorders including rheumatoid arthritis.

An approach to inhibit the action of cytokines, for example GH, prolactin or TNF, is the administration of antagonists.

One example of a GH antagonist is Pegvisomant, which is a modified GH molecule coated in polyethylene glycol (PEG). Pegvisomant has several beneficial effects, including, for example, decreased glomerular filtration rate due to an increased effective molecular weight, thereby reducing the dose required to produce the desired effect, [see Abuchowski et al J Biol Chem., 252, 3578-3581, (1977)]. However, a consequence of pegylation is a reduction in affinity of the modified GH molecule for GHR.

An example of a prolactin antagonist is disclosed in WO03/057729 (which is incorporated by reference in its entirety and more specifically the nucleotide and protein sequences encoding said prolactin antagonist). The prolactin antagonist comprises a modification to the human prolactin amino acid sequence that replaces a glycine residue at position 129 with an arginine residue. The modified prolactin protein acts as an inhibitor of prolactin receptor activation.

A number of therapeutic strategies to inhibit TNF have been developed on the basis of being able to i) inhibit TNF synthesis (e.g. using inhibitory cytokines, IL-10; thalidomide, corticosteroids, cyclosporin A; antisense oligonucleotides); ii) inhibition of TNF processing (e.g. metalloprotease (TACE) inhibitors); iii) neutralisation of TNF (e.g. using soluble TNF receptors or antibodies to TNF).

We describe polypeptides comprising multiple ligand binding domains of cytokine receptors and their use in the modulation of receptor mediated cytokine activation.

According to an aspect of the invention there is provided a polypeptide comprising at least two cytokine binding domains capable of binding to a cytokine receptor, wherein the domains are linked by a peptide linker molecule that comprises an inflexible helical region.

In a preferred embodiment of the invention said polypeptide acts as an antagonist of said cytokine receptor(s). Alternatively said polypeptide acts as an agonist. Preferably, the polypeptide comprises the domains in a tandem array. In preferred embodiments of the invention the polypeptide comprises 2, 3, 4, 5, 6, 7, 8, 9, or 10 domains in a tandem array.

In a still further preferred embodiment of the invention the polypeptide comprises more than 10 domains in a tandem array.

Preferably, the inflexible helical region comprises at least one copy of the motif A(EAAAK)ₓA, or a functional variant thereof. Preferably the peptide linker molecule comprises two copies of the motif EAAAK, with the length of the peptide linker molecule being extendible by the incremental addition of at least one amino acid.

A “functional variant” is a linker molecule that may differ in amino acid sequence by one or more substitutions, additions, deletions but that retains substantially a helical or non-helical conformation. Among preferred variants are those that vary from a reference amino acid sequence by conservative amino acid substitutions. Such substitutions are those that substitute a given amino acid by another amino acid of like characteristics. The following non-limiting list of amino acids are considered conservative replacements (similar): a) alanine, serine, and threonine; b) glutamic acid and aspartic acid; c) asparagine and glutamine d) arginine, lysine and histidine; e) isoleucine, leucine, methionine and valine and f) phenylalanine, tyrosine and tryptophan. Most highly preferred are amino acid substitutions that substantially maintain a flexible or an inflexible helical linker region.

In a further preferred embodiment of the invention the linker molecule comprises at least one flexible non-helical region.

Whilst the provision of the inflexible helical region maintains the spatial separation of the domains, as described above, the provision of a flexible non-helical region enables the domains to orientate into the binding sites of the cytokine receptor(s).

In one embodiment of the invention a flexible non-helical region is located at or near the amino-terminal end of the peptide linker molecule, thereby allowing the orientation of the binding domain located at the amino-terminal end of the peptide linker molecule in relation to its cognate receptor.

In a further embodiment of the invention the flexible non-helical region is located at or near the carboxyl-terminal end of the peptide linker molecule, thereby allowing the orientation of the binding domain located at the carboxyl-terminal end of the peptide linker molecule in relation to its cognate receptor.

In a still further embodiment of the invention the flexible non-helical region is located at or near the amino and the carboxyl-terminal end of the peptide linker molecule, thereby allowing the orientation of the binding domains positioned at the amino and carboxyl-terminal end respectively of the peptide linker molecule in relation to their cognate receptors.

Preferably the flexible non-helical region is located adjacent to at least one of the binding domains. Even more preferably the flexible non-helical region forms a junction between the binding domain and the inflexible helical region.

Even more preferably the inflexible helical region comprises at least one copy of the motif A(EAAAK)ₓA. The length of the inflexible non-helical region is extendable by increasing the number of repeats of this A(EAAAK)ₓA motif.

In a preferred embodiment of the invention, x in the A(EAAAK)ₓA motif is less than 10 copies. Even more preferably x is less than 5 copies. Even more preferably still x is selected from 1, 2, 3, 4 or 5 copies.

In a preferred embodiment of the invention there are no flexible connections between the rigid alpha helical linkers and the binding domains, but said binding domains are directly linked by the inflexible alpha helical linker.

In a preferred embodiment of the invention said binding domains are linked by a linking molecule consisting of an inflexible alpha helix.

In a preferred embodiment of the invention said helical linker molecule links the carboxyl terminus of one binding domain with the amino terminus of a second binding domain.

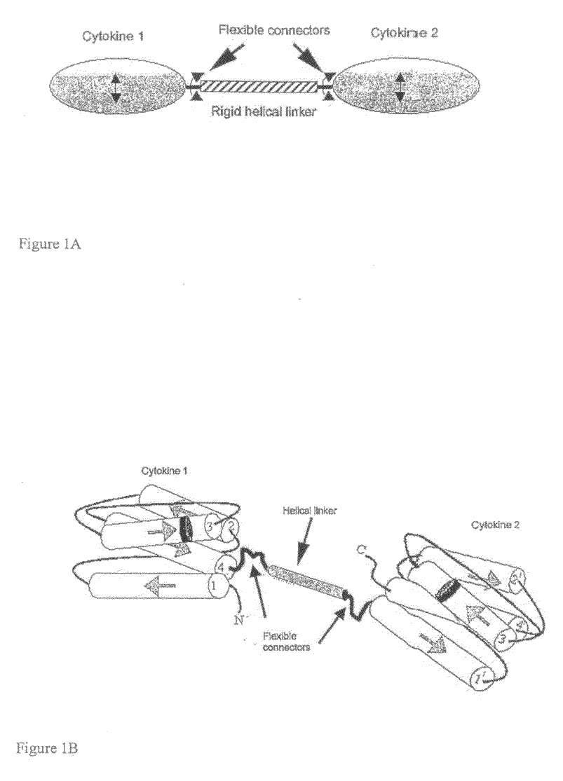

In this embodiment of the invention the helical linker is continuous between the C-terminal helix of the first cytokine molecule and the N-terminal helix of the second cytokine molecule, thus rigidly linking the two cytokine binding domains in a substantially fixed orientation. For example, this may involve the deletion of a short N-terminal and post-helical C-terminal region from a first cytokine domain and the short pre-helical N-terminal region from a second cytokine (i.e. residues 182-190 from a first cytokine, and residues 1-5 of a second cytokine as these are short regions of random coil conformation after the C-terminal helix (for example, helix 4 in FIG. 1B) in the first cytokine and before the N terminal (helix 1′ in FIG. 1B) of a second cytokine.

In different constructs this fixed orientation (both translational and rotational) can be altered either by the insertion of 1, 2, 3, 4, 5, 6, 7, 8, 9 or 10 additional amino acids, or by the deletion of 1, 2, 3, 4, 5, 6, 7, 8, 9 or 10 amino acids to produce molecules with novel properties, for example antagonistic properties. Addition of an extra amino acid will produce an additional relative translation of the two domains by approximately 1.5 Å and a relative rotation of the two domains around the helix axis of about +100°. Typically, linkers could start with two EAAAK units and will be lengthened by addition of A, AA, AAA, AAAA, EAAAA and EAAAK sequences.

In a further preferred embodiment of the invention the binding domains of the polypeptide are the same or similar to each other.

In still further embodiments of the invention the polypeptide comprises binding domains of cytokines selected from the group consisting of; growth hormone; leptin; erythropoietin; prolactin; interleukins (IL) IL-2, IL-3, IL-4, IL-5, IL-6, IL-7, IL-9, IL-10, IL-11, the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin (CT-1); leukocyte inhibitory factor (LIF); oncostatin M (OSM); interferon, IFNα and IFNγ; tumour necrosis factor (TNF)α, TNFβ, and RANK ligand.

In a further preferred embodiment of the invention at least one of the domains comprises a growth hormone binding domain.

In a further preferred embodiment of the invention said polypeptide comprises at least two binding domains of growth hormone, or a growth hormone variant.

Modified GH variants are disclosed in U.S. Pat. No. 5,849,535, that is incorporated by reference. The modification to GH is at both site 1 and site 2 binding sites. The modifications to site 1 produce a GH molecule that has a higher affinity for GHR compared to wild-type GH. These modified GH molecules act as agonists. There is also disclosure of site 2 modifications that result in the creation of GH antagonists. Further examples of modifications to GH that alter the binding affinity of GH for site 1 are disclosed in U.S. Pat. No. 5,854,026; U.S. Pat. No. 6,004,931; U.S. Pat. No. 6,022,711; U.S. Pat. No. 6,057,292; and U.S. Pat. No. 6,136,563 each of which are incorporated by reference. Modifications to site 2 are also described, in particular amino acid residue G120 in GH which when modified to either arginine, lysine, tryptophan, tyrosine, phenylalanine, or glutamic acid creates a GH molecule with antagonistic properties.

In our co-pending application WO03/070765, which is incorporated by reference, we describe the fusion of a GH variant with antagonistic activity with respect to GH receptor activation. The GH variant is fused via a flexible linker to the extracellular domain of the growth hormone receptor. This chimeric polypeptide shows delayed clearance and antagonistic activity. The provision of a similar chimeric polypeptide but with an inflexible or partially flexible linker is also within the scope of the invention herein disclosed.

In an alternative preferred embodiment of the invention said polypeptide comprises at least two binding domains of prolactin, or a prolactin variant.

In a preferred embodiment of the invention said prolactin variant polypeptide comprises an amino acid sequence wherein said amino acid sequence is modified at position 129 of human prolactin.

In a preferred embodiment of the invention said modification is an amino acid substitution. Preferably said substitution replaces a glycine amino acid residue with an arginine amino acid residue. Preferably said modification further comprises the deletion of at least 9, 10, 11, 12, 13 or 14 amino terminal amino acid residues.

In an alternative embodiment of the invention the binding domains of the polypeptide are dissimilar to each other.

In a preferred embodiment of the invention said polypeptide comprises a first binding domain that is a growth hormone binding domain and a second binding domain that is a prolactin binding domain.

Preferably said polypeptide consists of a growth hormone binding domain and a prolactin binding domain.

In an alternative preferred embodiment of the invention said polypeptide comprises a first binding domain that is a modified growth hormone binding domain and a second binding domain that is a modified prolactin binding domain.

Preferably said polypeptide consists of a modified growth hormone binding domain and a modified prolactin binding domain.

In a preferred embodiment of the invention said modified growth hormone binding domain comprises an amino acid substitution at amino acid position glycine 120. Preferably, said modification is a substitution of glycine 120 for an amino acid selected from the group consisting of arginine, lysine, tryptophan, tyrosine, phenylalanine, or glutamic acid.

In a preferred embodiment of the invention said modification is the substitution of glycine 120 with an arginine amino acid residue.

In a further preferred embodiment of the invention said modified prolactin binding domain comprises a modification of glycine 129. Preferably said modification is the substitution of glycine 129 with an arginine amino acid residue. Preferably said modification further comprises the deletion of at least 9, 10, 11, 12, 13 or 14 amino terminal amino acid residues.

In a further preferred embodiment of the invention said polypeptide further comprises a ligand binding domain of a cytokine receptor. Preferably said receptor is a growth hormone receptor. In an alternative preferred embodiment of the invention said receptor is a prolactin receptor.

In a preferred embodiment of the invention said ligand binding domain may be linked to said cytokine binding domain by a linker comprising or consisting of an inflexible helical region.

According to a further aspect of the invention there is provided a nucleic acid molecule that encodes a polypeptide according to the invention.

In a preferred embodiment of the invention said nucleic acid molecule is a vector adapted for expression of said polypeptide.

Typically, the adaptation includes the provision of transcription control sequences (promoter sequences) that mediate cell/tissue specific expression. These promoter sequences may be cell/tissue specific, inducible or constitutive.

Promoter is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences and are therefore position independent). Enhancers function to increase the rate of transcription of the gene to which the enhancer is linked. Enhancer activity is responsive to trans acting transcription factors (polypeptides) that have been shown to bind specifically to enhancer elements. The binding/activity of transcription factors (please see Eukaryotic Transcription Factors, by David S Latchman, Academic Press Ltd, San Diego) is responsive to a number of environmental cues that include, by example, intermediary metabolites (e.g. glucose), environmental effectors (e.g. heat).

Promoter elements also include so called TATA box and RNA polymerase initiation selection (RIS) sequences that function to select a site of transcription initiation. These sequences also bind polypeptides that function, inter alia, to facilitate transcription initiation selection by RNA polymerase.

Adaptations also include the provision of selectable markers and autonomous replication sequences which both facilitate the maintenance of the vector in the eukaryotic or prokaryotic cell. Vectors that are maintained autonomously are referred to as episomal vectors.

Adaptations which facilitate the expression of vector encoded genes include the provision of transcription termination/polyadenylation sequences. This also includes the provision of internal ribosome entry sites (IRES) that function to maximise expression of vector encoded genes arranged in bicistronic or multi-cistronic expression cassettes.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, N.Y. and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

It will be apparent to one skilled in the art that the vectors according to the invention could be gene therapy vectors. Gene therapy vectors are typically viral based. A number of viruses are commonly used as vectors for the delivery of exogenous genes. Commonly employed vectors include recombinantly modified enveloped or non-enveloped DNA and RNA viruses, preferably selected from baculoviridiae, parvoviridiae, picomoviridiae, herpesveridiae, poxyiridae, adenoviridiae, or picornnaviridiae. Chimeric vectors may also be employed which exploit advantageous elements of each of the parent vector properties (See e.g., Feng, et al. (1997) Nature Biotechnology 15:866-870). Such viral vectors may be wild-type or may be modified by recombinant DNA techniques to be replication deficient, conditionally replicating or replication competent.

Preferred vectors are derived from the adenoviral, adeno-associated viral and retroviral genomes. In the most preferred practice of the invention, the vectors are derived from the human adenovirus genome. Particularly preferred vectors are derived from the human adenovirus serotypes 2 or 5. The replicative capacity of such vectors may be attenuated (to the point of being considered “replication deficient”) by modifications or deletions in the E1a and/or E1b coding regions. Other modifications to the viral genome to achieve particular expression characteristics or permit repeat administration or lower immune response are preferred.

Alternatively, the viral vectors may be conditionally replicating or replication competent. Conditionally replicating viral vectors are used to achieve selective expression in particular cell types while avoiding untoward broad spectrum infection. Examples of conditionally replicating vectors are described in Pennisi, E. (1996) Science 274:342-343; Russell, and S. J. (1994) Eur. J. of Cancer 30A(8):1165-1171. Additional examples of selectively replicating vectors include those vectors wherein an gene essential for replication of the virus is under control of a promoter which is active only in a particular cell type or cell state such that in the absence of expression of such gene, the virus will not replicate. Examples of such vectors are described in Henderson, et al., U.S. Pat. No. 5,698,443 issued Dec. 16, 1997 and Henderson, et al., U.S. Pat. No. 5,871,726 issued Feb. 16, 1999 the entire teachings of which are herein incorporated by reference.

Additionally, the viral genome may be modified to include inducible promoters that achieve replication or expression only under certain conditions. Examples of inducible promoters are known in the scientific literature (See, e.g. Yoshida and Hamada (1997) Biochem. Biophys. Res. Comm. 230:426-430; Iida, et al. (1996) J. Virol. 70(9):6054-6059; Hwang, et al. (1997) J. Virol 71(9):7128-7131; Lee, et al. (1997) Mol. Cell. Biol. 17(9):5097-5105; and Dreher, et al. (1997) J. Biol. Chem 272(46); 29364-29371.

Vectors may also be non-viral and are available from a number of commercial sources readily available to the person skilled in the art. For example, the vectors may be plasmids that can be episomal or integrating.

According to a further aspect of the invention there is provided a cell transformed or transfected with the nucleic acid or vector according to the invention.

In a preferred embodiment of the invention said cell is a eukaryotic cell.

Preferably said eukaryotic cell is selected from the group consisting of: a fungal cell e.g. Saccharomyces cerevisiae, Pichia spp; slime mold (e.g. Dictyostelium spp); insect cell (e.g. Spodoptera frugiperda); a plant cell; or a mammalian cell (e.g. CHO cell).

In an alternative preferred embodiment of the invention said cell is a prokaryotic cell.

In a further aspect of the invention there is provided a method to prepare a polypeptide according to the invention said method comprising the steps of;
i) growing a cell according to the invention in conditions conducive to the production of a polypeptide according to the invention; and
ii) isolating the polypeptide from the cell, or its growth environment.

In a preferred method of the invention said polypeptide is provided with an affinity tag.

Affinity tags are known in the art and include, maltose binding protein, glutathione S transferase, calmodulin binding protein and the engineering of polyhistidine tracks into proteins that are then purified by affinity purification on nickel containing matrices. In many cases commercially available vectors and/or kits can be used to fuse a protein of interest to a suitable affinity tag that is subsequently transfected into a host cell for expression and subsequent extraction and purification on an affinity matrix.

In our co-pending application, WO 03/034275, the content of which is incorporated by reference, we describe a novel affinity tag for polypeptides that utilises a domain that includes a signal sequence that directs the addition of glycosylphosphatidylinositol to the polypeptide. Polypeptides that include a glycosylphosphatidylinositol tag preferentially insert into lipid membranes and can have antagonistic effects on cytokine receptor activation. Therefore, the invention herein disclosed encompasses polypeptides with an attached glycosylphosphatidylinositol molecule.

According to a further aspect of the invention there is provided a polypeptide comprising a first cytokine binding domain linked to a second cytokine binding domain wherein said polypeptide further comprises an extracellular domain of a cytokine receptor.

In a preferred embodiment of the invention said first and second binding domains are linked by a flexible linker molecule.

In an alternative preferred embodiment of the invention said first and second binding domains are linked by a peptide linker molecule that comprises an inflexible helical region.

In a preferred embodiment of the invention said first and second binding domains are linked by a peptide linker molecule comprising an inflexible helical region and a flexible, non-helical region.

Peptide linkers that comprise inflexible helical regions and combinations of inflexible helical regions and flexible, non-helical regions have been described previously above and are applicable to this embodiment of the invention as are the previously specified cytokines and cytokine receptors.

In a preferred embodiment of the invention the extracellular domain of the cytokine receptor is linked to the first or second cytokine binding domains via a linker molecule. Preferably said linker molecule comprises an inflexible helical region.

In an alternative preferred embodiment of the invention said linker molecule is flexible.

Preferably said linker molecule comprises an inflexible helical region and a flexible, non-helical region.

In a preferred embodiment of the invention said cytokine binding domain is growth hormone, or a growth hormone variant thereof, and said extracellular domain is a growth hormone extracellular domain. Preferably said domains are human.

The polypeptide of the invention may demonstrate dual functionality. Firstly, the first and second domains comprising cytokines, or parts thereof, which are preferably linked by a peptide linker molecule comprising an inflexible helical region, are capable of binding to cell surface cytokine receptors and sterically hinder the association of these receptors into receptor complexes, thus preventing downstream cell signalling. Secondly, the provision of a third domain comprising cytokine receptors, or parts thereof, are capable of functioning as a soluble receptor, thus ligating any cytokine, prior to its binding to the cell surface receptor. This third domain is preferably linked to the first or second domain by a peptide linker molecule comprising an inflexible helical region. In an alternative embodiment of the invention the third domain is preferably linked to the first or second domain by a peptide linker molecule comprising a flexible non-helical region.

In a preferred embodiment of the invention said peptide linker molecules further comprise an amino acid sequence that is sensitive to proteolytic cleavage.

According to a further aspect of the invention there is provided the use a polypeptide or nucleic acid molecule according to the invention as a pharmaceutical.

Preferably there is provided a pharmaceutical composition comprising the polypeptide or nucleic acid molecule according to the invention. Preferably said pharmaceutical composition comprises a carrier, excipient, and/or diluent.

When administered, the therapeutic compositions of the present invention are administered in pharmaceutically acceptable preparations. The term “pharmaceutically acceptable” means a non-toxic material that does not interfere with the effectiveness of the biological activity of the active ingredients. Such preparations may routinely contain salts, buffering agents, compatible carriers, preservatives and optionally other therapeutic agents.

When used in medicine, the salts should be pharmaceutically acceptable, but non-pharmaceutically acceptable salts may conveniently be used to prepare pharmaceutically-acceptable salts thereof and are not excluded from the scope of the invention. Such pharmacologically and pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. Also, pharmaceutically-acceptable salts can be prepared as alkaline metal or alkaline earth salts, such as sodium, potassium or calcium salts.

The pharmaceutical compositions may contain suitable buffering agents, including: acetic acid in a salt; citric acid in a salt; boric acid in a salt; and phosphoric acid in a salt.

The compositions may be combined, if desired, with a pharmaceutically-acceptable carrier. The term “pharmaceutically-acceptable carrier” as used herein means one or more compatible solid or liquid fillers, diluents or encapsulating substances that are suitable for administration into a human. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction that would substantially impair the desired pharmaceutical efficacy.

The pharmaceutical compositions may conveniently be presented in unit dosage form and may be prepared by any of the methods well-known in the art of pharmacy. All methods include the step of bringing the active agent into association with a carrier that constitutes one or more accessory ingredients. In general, the compositions are prepared by uniformly and intimately bringing the active compound into association with a liquid carrier, a finely divided solid carrier, or both, and then, if necessary, shaping the product.

The pharmaceutical compositions also may contain, optionally, suitable preservatives, such as: benzalkonium chloride; chlorobutanol; parabens and thimerosal.

The pharmaceutical compositions of the invention can be administered by any conventional route, including injection or by gradual infusion over time. The administration may, for example, be oral, intravenous, intraperitoneal, intramuscular, intracavity, subcutaneous, or transdermal.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

The compositions of the invention are administered in effective amounts. An “effective amount” is that amount of a composition that alone, or together with further doses, produces the desired response. In the case of treating a particular disease, such as cancer, the desired response is inhibiting the progression of the disease. This may involve only slowing the progression of the disease temporarily, although more preferably, it involves halting the progression of the disease permanently. This can be monitored by routine methods.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In a further aspect of the invention there is provided the use of a polypeptide or nucleic acid molecule according to the invention for the manufacture of a medicament for the treatment of a disease selected from the group consisting of; acromegaly; gigantism; GH deficiency; Turners Syndrome; renal failure; osteoporosis; osteoarthritis; diabetes mellitus; cancer (for example, prostate cancer, a cervical cancer, a breast cancer, melanoma, hepatoma, renal cancer, glioma, bladder cancer, lung cancer, neural cancer, ovarian cancer, testicular cancer, pancreatic cancer, gastrointestinal cancer, lymphoma); obesity; insulin resistance; hyperlipidaemia; hypertension; anaemia; autoimmune and infectious disease; inflammatory disorders including rheumatoid arthritis.

The invention also provides for a method of treating a human or animal subject comprising administering an effective amount of the polypeptide, nucleic acid molecule, pharmaceutical composition or medicament to the subject.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Embodiments of the invention will now be described by way of example only and with reference to the following figures wherein;

FIG. 1A The cytokine domains (ovals) are connected by an alpha helix (shaded rectangle). Flexible linkers (curved arrows) connect the first cytokine domain to the helix and the helix to the second cytokine domain; FIG. 1B. The cytokine domains are connected by an alpha helix. Flexible linkers connect the first cytokine domain to the helical linker and the helical linker to the second cytokine domain;

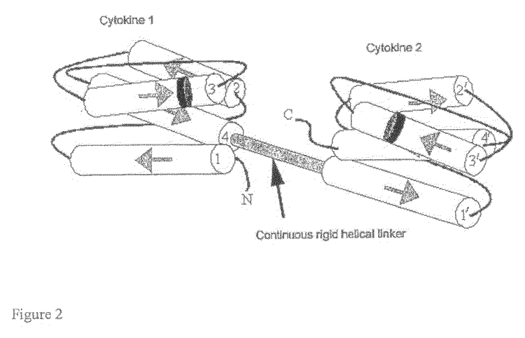

FIG. 2. The helical linker has no flexible connectors—instead it continues the C-terminal helix (4) of cytokine 1 and joins it to the N-terminal helix (1′) of cytokine 2 to form a rigid tandem linked by a single long helix 4-linker helix-1′. The relative orientation of the two cytokine domains is therefore fixed. However by making different constructs by adding or removing amino acids from the linker it is possible to generate a series of rigid tandems in which the domains are differently oriented;

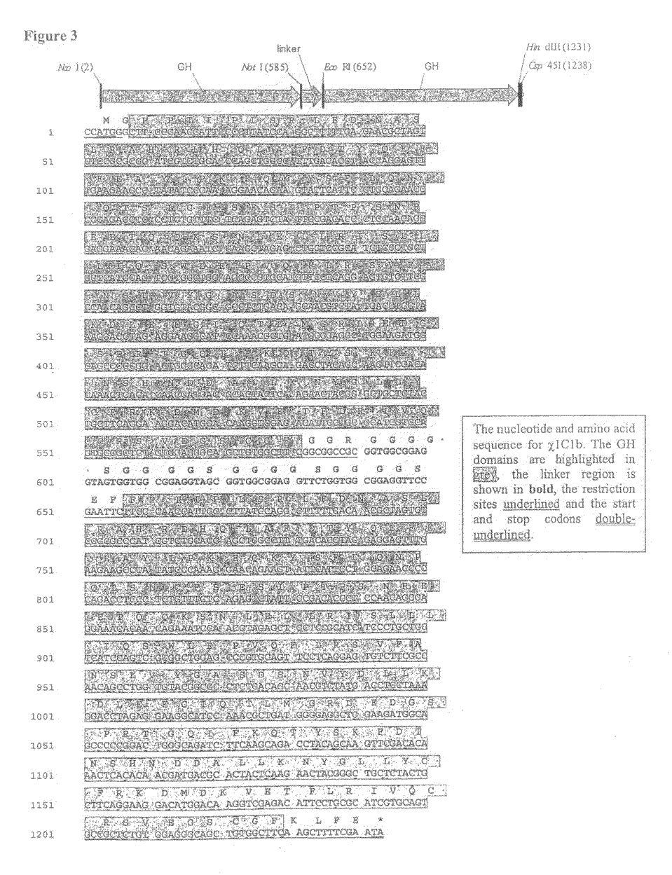

FIG. 3 illustrates the map and nucleotide/amino acid sequence for construct χ1C1b;

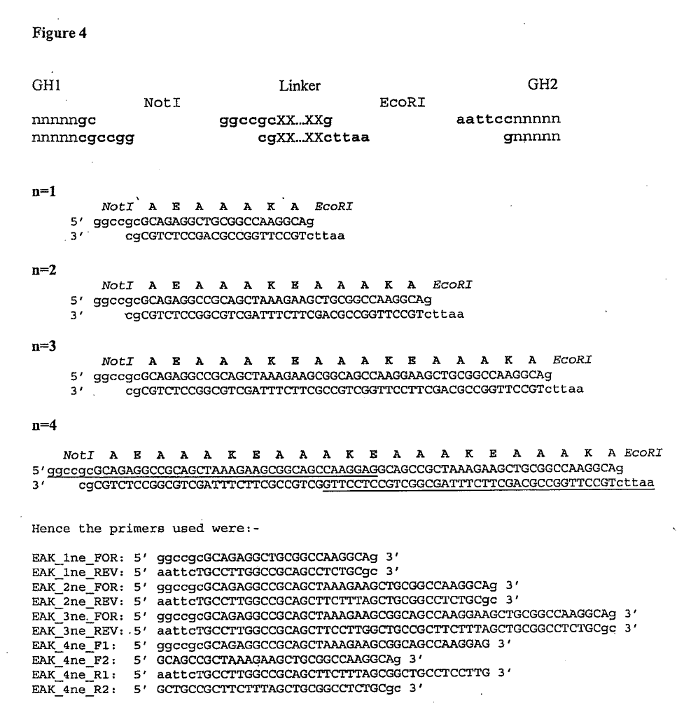

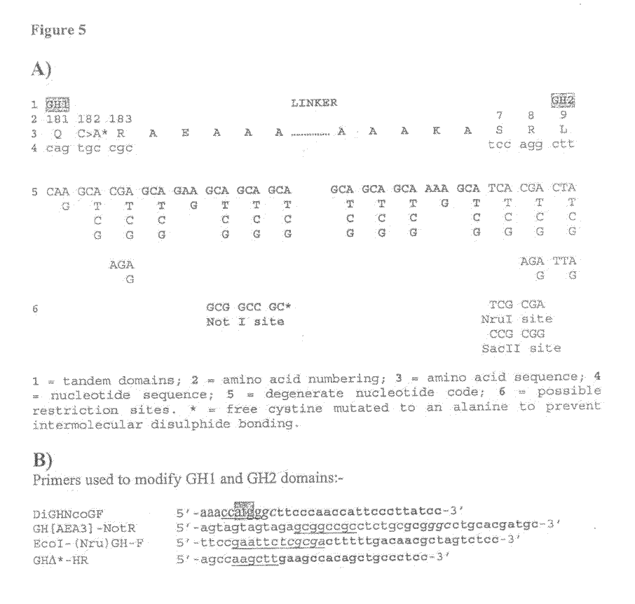

FIG. 4 illustrates an overview of the linker design and primers used to generate tandems with helical linkers with flexible ends;

FIG. 5 illustrates, A) Design of the boundary regions between the GH domains and the linker to allow ligation of primer duplexes to produce uninterrupted helical linkers between the domains. B) The primers used to modify χ1C1b to generate χ1C5;

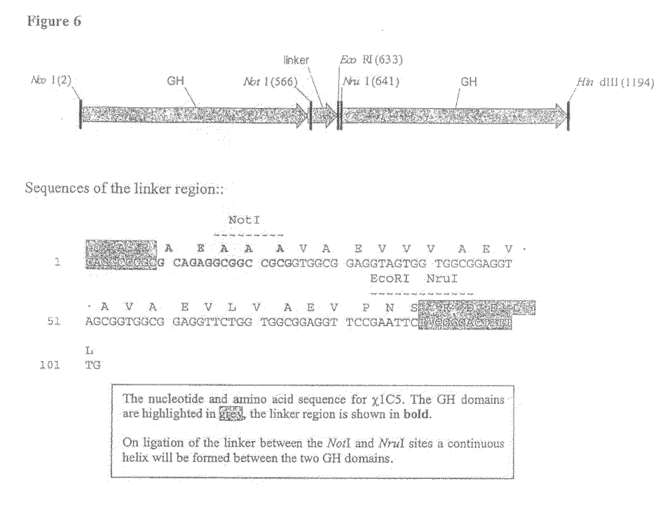

FIG. 6 illustrates a map of construct χ1C5 and the sequence of the linker region;

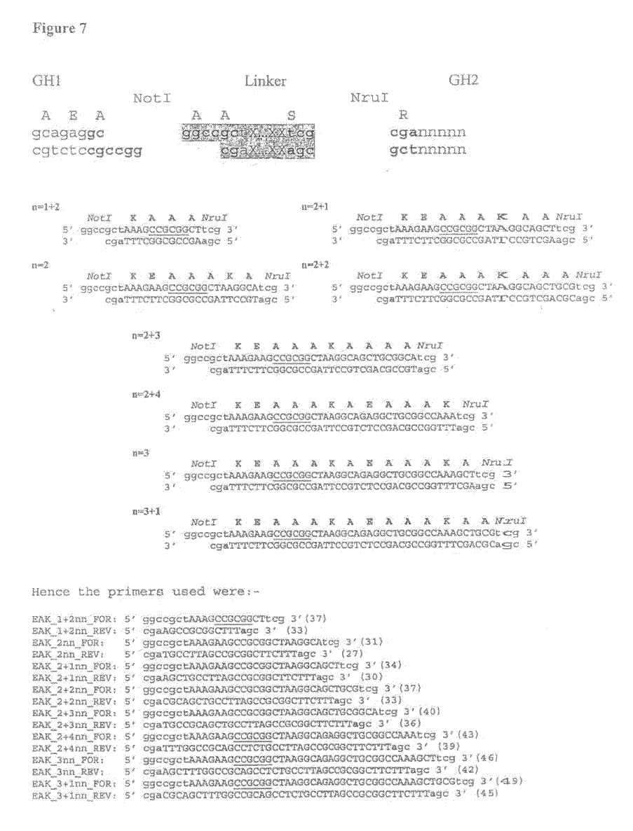

FIG. 7 illustrates an overview of the linker design and primers used to generate tandems with rigid helical linkers;

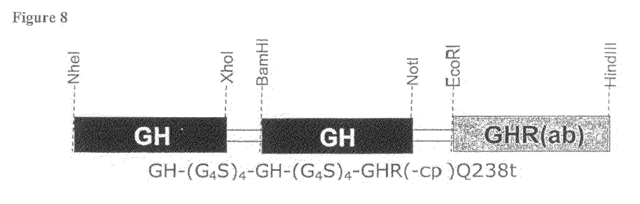

FIG. 8 illustrates a schematic diagram showing the strategy for the construction of χ1L1;

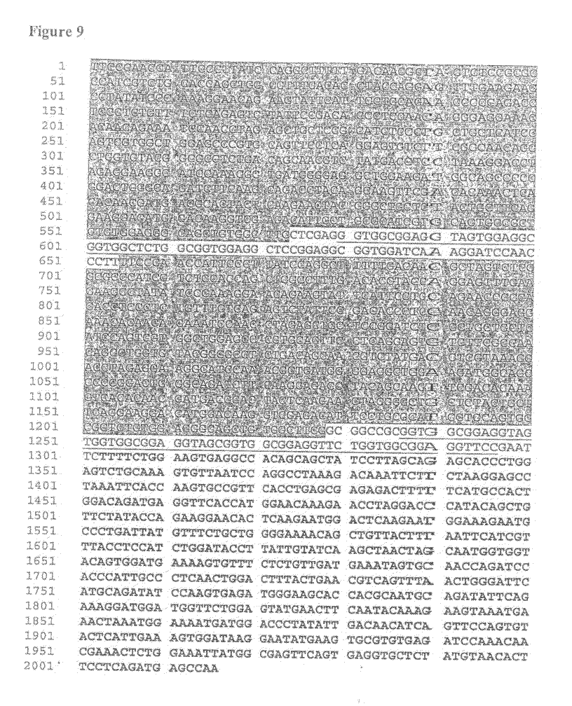

FIG. 9 illustrates the nucleotide sequence of χ1L1. The GH domains are shown in grey, the GHR domain in bold and the linkers underlined:

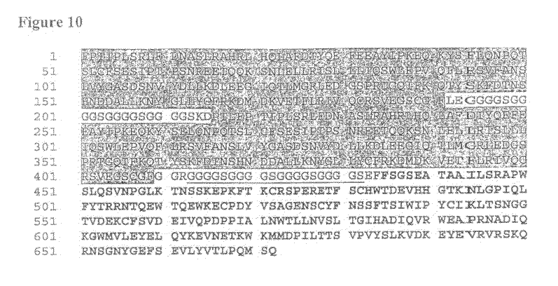

FIG. 10 illustrates the amino acid sequence of χ1L1. The GH domains are shown in grey, the GHR domain in bold and the linkers underlined;

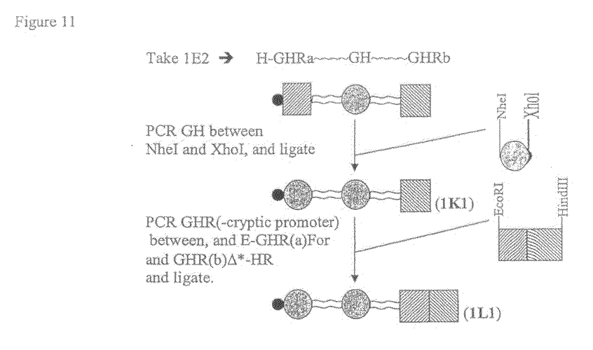

FIG. 11 illustrates a schematic diagram showing the cloning strategy for the construction of χ1L1;

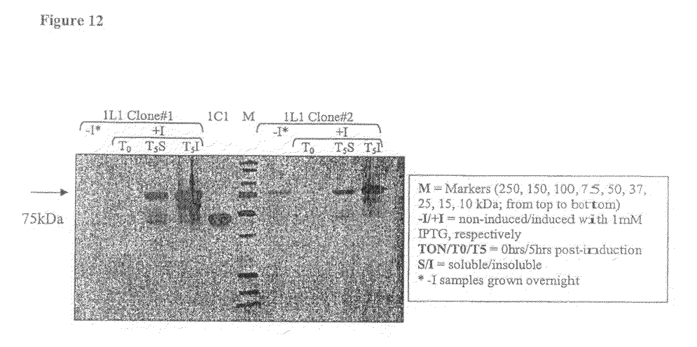

FIG. 12 illustrates the expression of χ1L1;

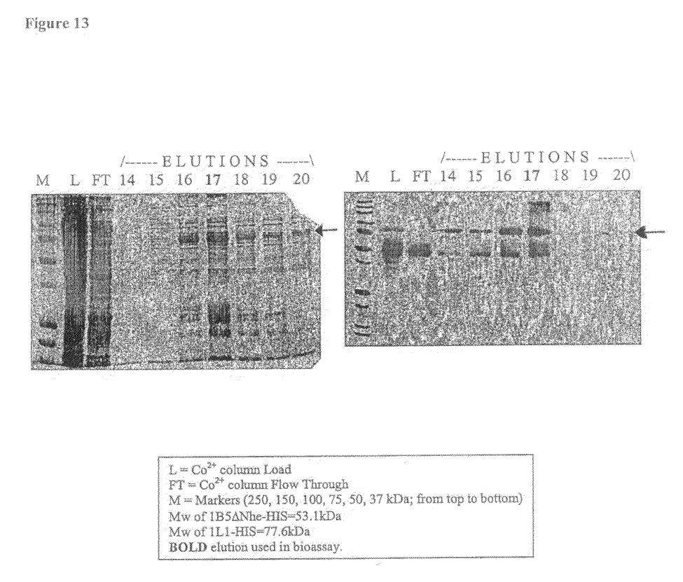

FIG. 13 illustrates a preliminary purification of χ1L1-His χ1L1-His was purified using a Co²⁺-column;

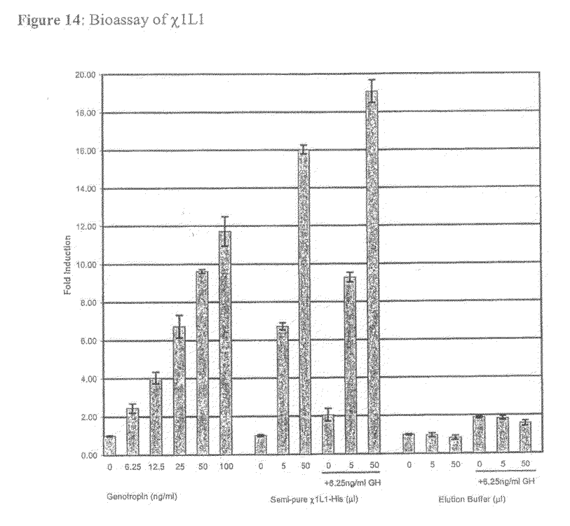

FIG. 14 illustrates that χ1L1 shows agonistic activity;

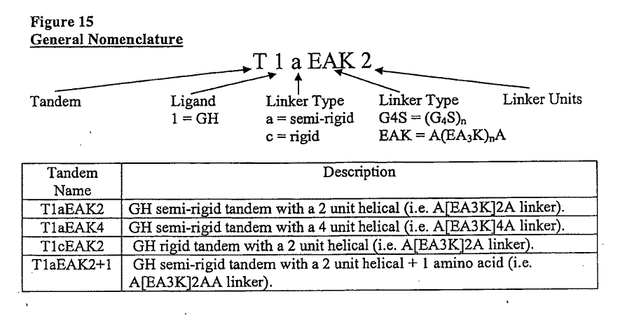

FIG. 15 summarises the nomenclature used with respect to rigid or semi-rigid GH constructs;

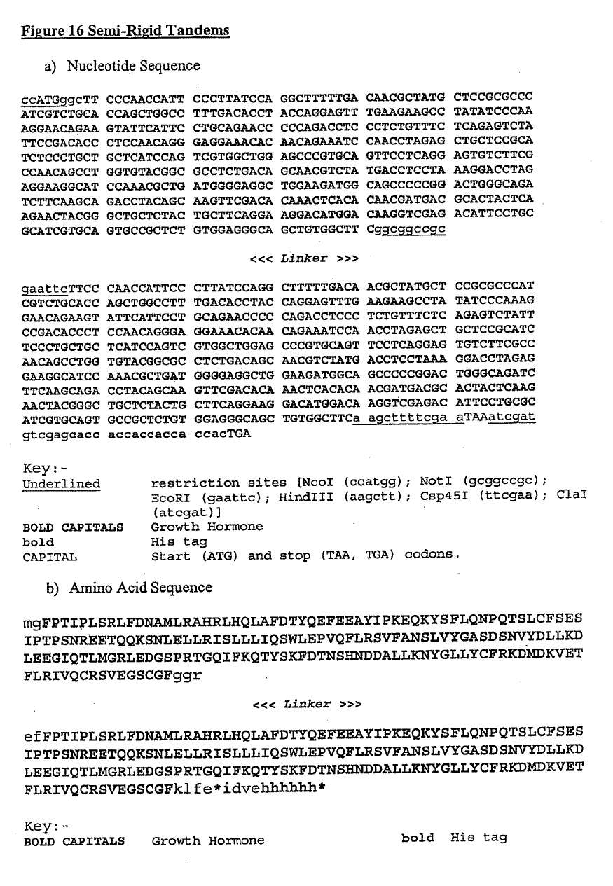

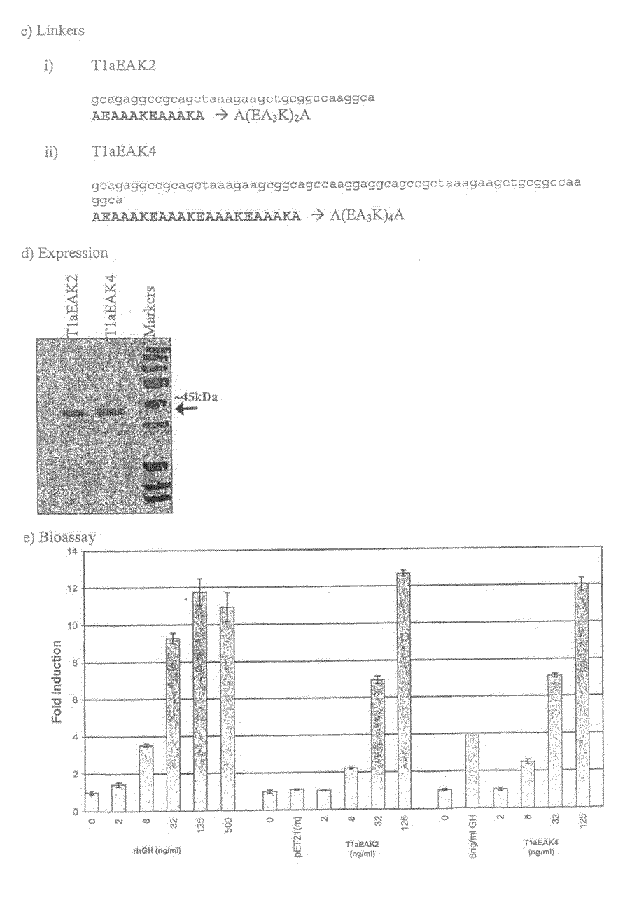

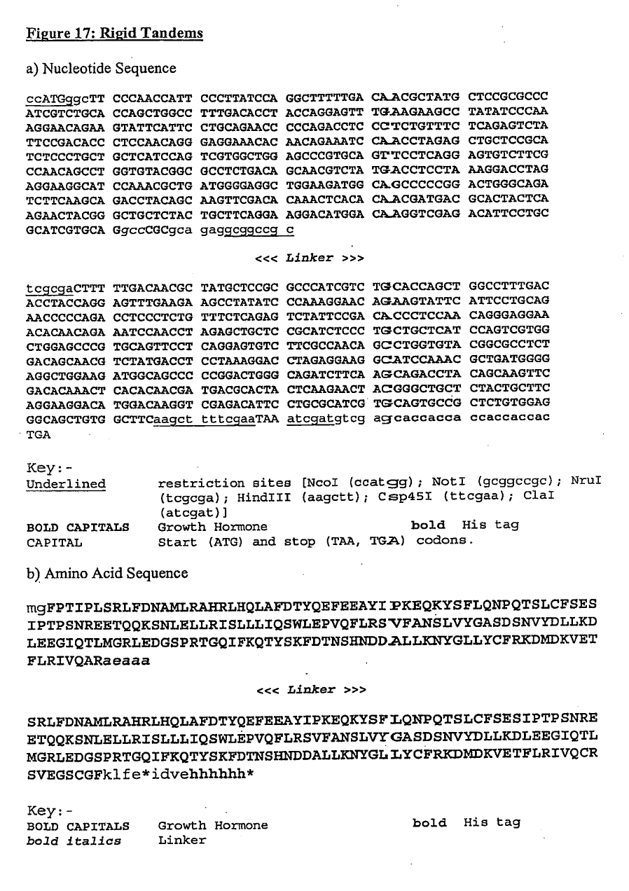

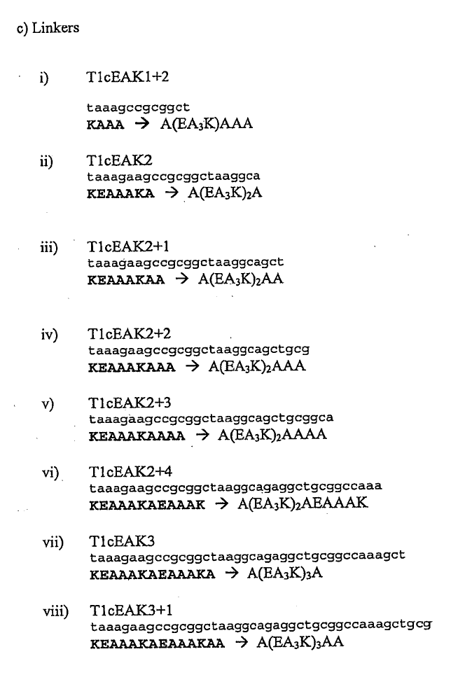

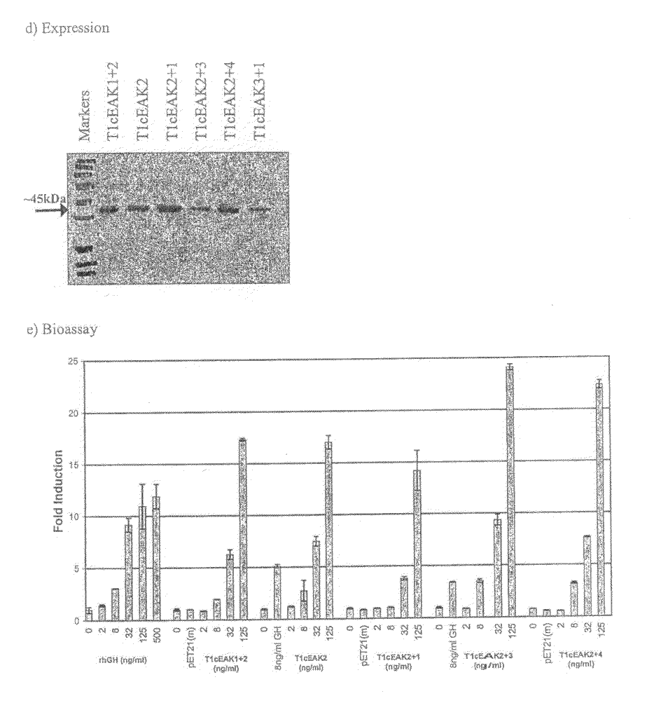

FIG. 16 a) is the nucleic acid sequence of a GH tandem comprising a semi-rigid linker sequence; b) is the amino acid sequence of a GH tandem comprising a semi-rigid linker sequence; c) illustrates examples of semi-rigid linkers used in the construction of GH tandems; d) illustrates bacterial expression of GH tandems comprising semi-rigid linkers; and e) illustrates the bioactivity of GH tandems comprising semi-rigid linkers; and

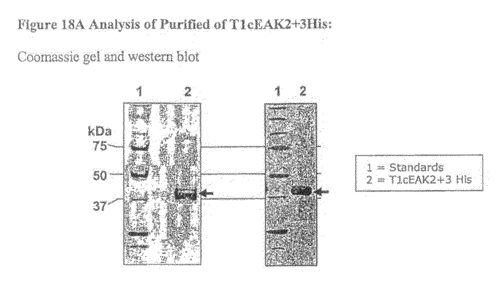

FIG. 17 a) is the nucleic acid sequence of a GH tandem comprising a rigid linker sequence; b) is the amino acid sequence of a GH tandem comprising a rigid linker sequence; c) illustrates examples of rigid linkers used in the construction of GH tandems; d) illustrates bacterial expression of GH tandems comprising rigid linkers; and e) illustrates the bioactivity of GH tandems comprising rigid linkers.

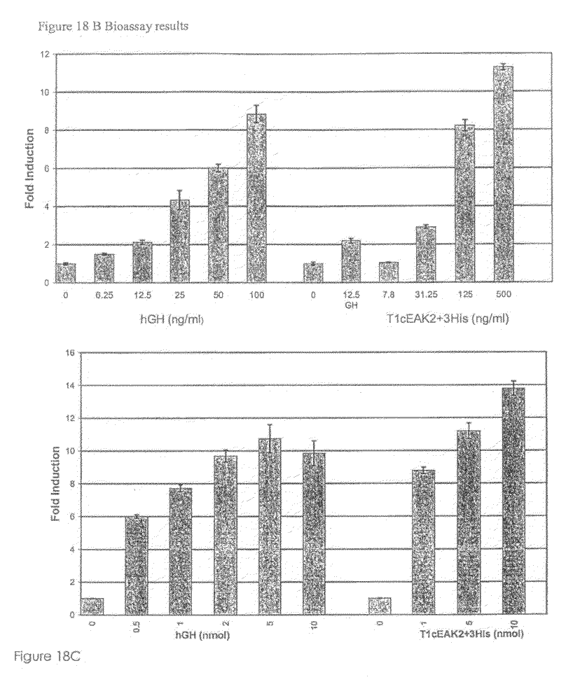

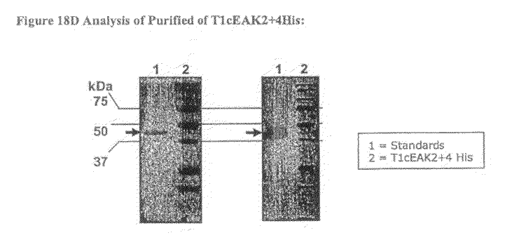

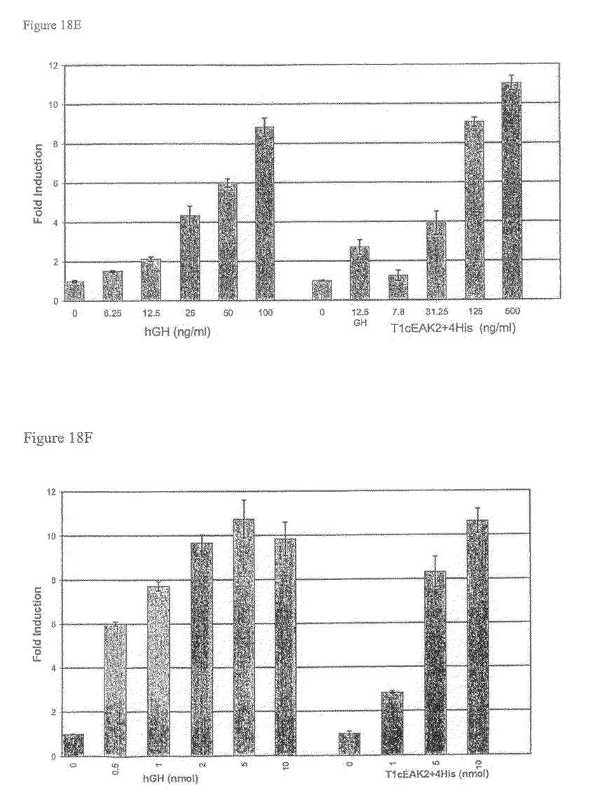

FIG. 18A illustrates the purification of T1cEAK2+3his and analysis by coomassie staining and western blot; FIGS. 18B and 18C illustrates the bioactivity of T1cEAK2+3his; FIG. 18D illustrates the purification of T1cEAK2+4his and analysis by coomassie staining and western blot; and FIGS. 18E and 18F illustrates the bioactivity of T1cEAK2+4his;

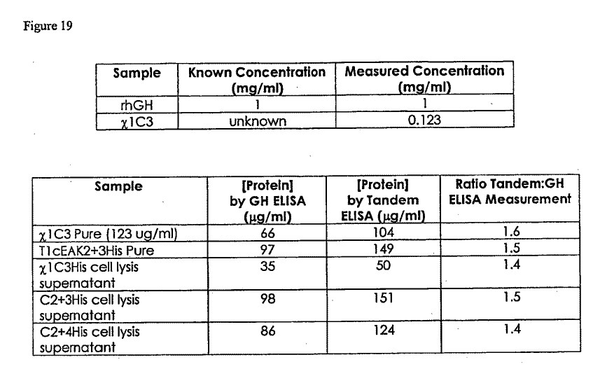

FIG. 19 illustrates an ELISA for detection of growth hormone tandems;

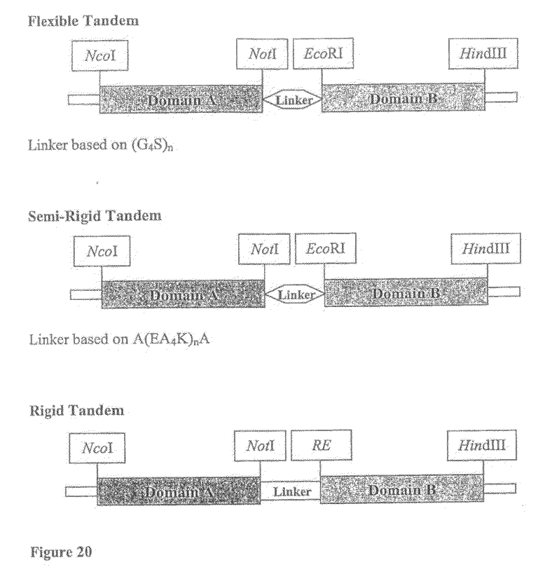

FIG. 20 is a schematic illustration of growth hormone tandems linked by flexible, semi-rigid and rigid linkers;

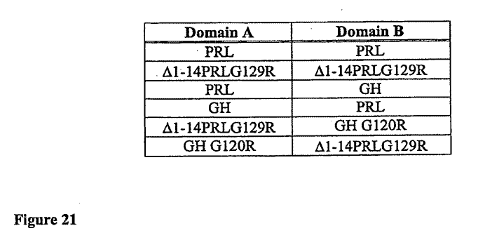

FIG. 21 illustrates examples of possible combinations of prolactin (PRL), growth hormone (GH) and their antagonistic mutants;

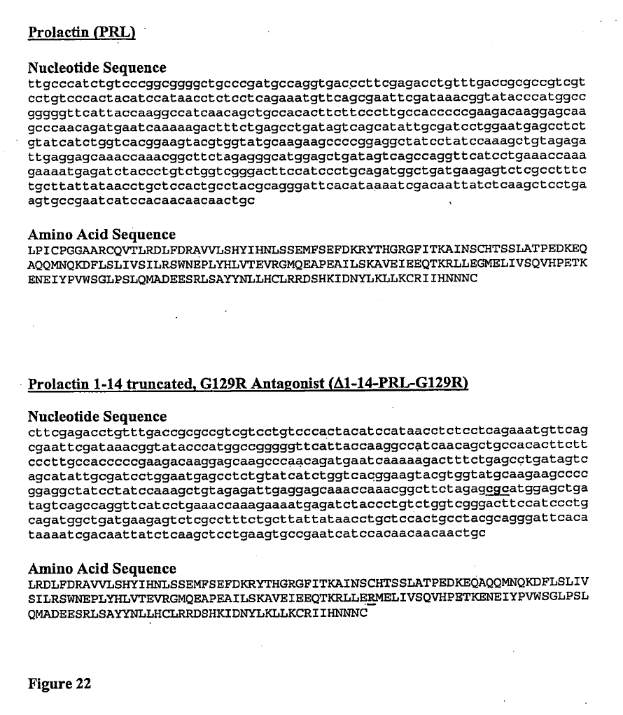

FIG. 22 illustrates the nucleotide and amino acid sequences of prolactin and one of its antagonistic forms, the 1-14 amino acid truncated G129R mutant (underlined);

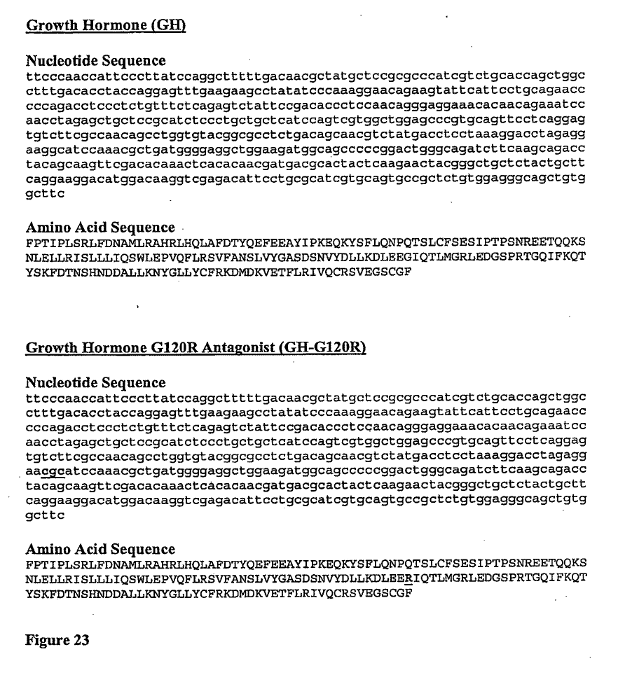

FIG. 23 illustrates the nucleotide and amino acid sequences of growth hormone and its antagonistic form, the G120R mutant (underlined);

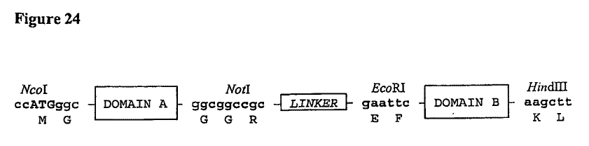

FIG. 24 is a schematic illustration of a prolactin tandem; and

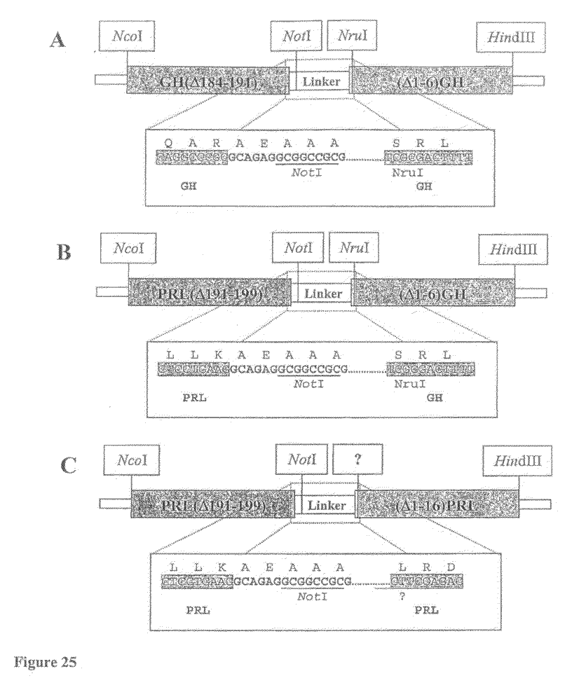

FIG. 25 (A) a schematic of the GH rigid-tandem constructs with the engineered restriction sites, NotI and NruI, which allow connection of the linker directly to the terminal helices of the neighbouring domains and also facilitates the variation of linker. (B) a schematic of the PRL-linker-GH rigid construct with the engineered restriction sites, NotI and NruI, which have similar functions as in (A), the NotI site is in the linker regions and so can just be appended to the truncated PRL gene in domain A. (C) a schematic diagram of the PRL rigid-tandem, a unique restriction site needs to be engineered, using the degenerate amino acid code, at the boundary between the linker and the PRL in domain B to enable easy synthesis and modification of the tandem gene.

### MATERIALS AND METHODS

The modification of the linker in the GH tandem was initiated from the gene for the GH-(G₄S)-GH molecule (χ1C1) which had been modified to remove a 30 amino acid overhang from the N-terminus of the expressed protein, the gene was also subcloned into a modified pET21 (+) vector, to give pET21:χ1C1 (FIG. 3).

For the constructs with the helical linkers with flexible ends linker was constructed by ligating together complementary oligonucleotides; these oligonucleotides were designed to encode the desired linker and to have ends which would ligate into the vector, pET21:χ1C1b, which had been digested with NotI and EcoRI. An overview of these linkers is shown in FIG. 4.

For the rigid linker between the GH domains, the GH domains had to be modified to truncate their C-terminus (GH1) and N-terminus (GH2) so that the domains ended at the end of the helices. Restriction sites were then designed, utilising degenerate codon usage, which would enable the new linkers to be introduced without any interruption to the helix that would be formed between the two domains (FIG. 5A). Primers were designed to carry out these modifications to the GH domains of the GH-tandem (FIG. 5b). The resultant construct was designated χ1C5 (FIG. 6). Complementary oligonucleotides were used to generate the linker region flanked by a NotI and NruI sites, these were then ligated into pET21:χ1C5 which had been digested with NotI and NruI. An overview of these linkers is shown in FIG. 7.

Expression of the constructs will be carried out by first transforming the pET expression vector into the expression strain, E. coli BL21(DE3) CodonPlus RIPL. Expression maybe carried under a number of different conditions which include different incubation temperatures (e.g. room temperature, 37° C.), different media (e.g. LB, 2YT, 5YT, etc.), different induction points (i.e. OD₆₀₀ at which the culture is induced), different concentrations of IPTG (or other inducer) used to induce the culture, and the time at which the cells are harvested post-induction.

A His-tag can be added to the C-terminus of the construct which would facilitate its purification using immobilised metal-ion affinity chromatography (with Ni²⁺ or Co²⁺ columns). Constructs which do not have a His-tag maybe purified using a variety of means such as ion-exchange chromatography, hydrophobic columns and size-exclusion chromatography. One or more of these purification techniques maybe required to produce protein of a suitable purity.

### Construction of χ1C5

Modified GH domains were generated using PCR and the relevant primers. GH1 was modified using DiGHNcoGF and GH[AEA3]NotR and GH2 modified with EcoI-(Nru)GH-F and GHΔ*-HR. The PCR reactions consisted off; 1 μl 100 pmol/μl forward primer, 1 μl 100 pmol/μl reverse primer, 1 μl pTrcHisGHstop (dilute), 1 μl 10 mM dNTPs, 5 μl 10× amplification buffer, 1 μl 50 mM MgSO₄, 0.5 μl Pfx polymerase, 39.5 μl sterile water. PCR was performed on these reaction mixes using the following thermal profile; 95° C. for 5 min; 15× (95° C. for 45 sec., 55° C. for 45 sec., 72° C. for 45 sec.); 72° C. for 5 min. The PCR products were verified using an agarose gel and the desired PCR product purified. Modified GH1 was ligated into pET21:χ1C1b between the NcoI and NotI sites to give pET21:χ1C4. Modified GH2 was ligated into pET21:χ1C4 between the EcoRI and HindIII sites to give pET21:χ1C5. An overview of this process is shown in FIG. 8.

### Linker Variation

### Phosphorylation of Primers

When 2 or more primer duplexes were required to generate the linker, the primers containing the internal 5′-end were first phosphorylated. The following reaction mix was made for each primer to be phosphorylated: 2 μl 100 pmol/μl oligonucleotides, 2 μl 10× Kinase Buffer, 2 μl 10 mM ATP, 13 μl sterile water, 1 μl T4 polynucleotide kinase (10 U/μl). These were incubated for 30 min at 37° C. and then at 70° C. for 10 min. The samples were then diluted 1:10 using Annealing Buffer (10 mM TRIS, 50 mM NaCl, 1 mM EDTA, pH 7.5-8.0) to obtain a solution of 0.1 pmol/μl. These could then be used in the annealing reaction, below.

### Annealing of the Primer Duplexes

The primers were diluted to 0.1 pmol/μl using Annealing Buffer (10 mM TRIS, 50 mM NaCl, 1 mM EDTA, pH 7.5-8.0). 10 μl of complementary primers were mixed in a fresh tube. The tube was then incubated at 95° C. for 2 min and the temperature allowed to drop to 30° C. over a period of 40-60 min. In the cases where more than one primer duplex was required equal volumes of the primer duplexes were mixed to provide a solution containing all the primer duplexes needed to form the desired linker. The solutions were then kept on ice.

### Ligation and Transformation

Approximately 200 ng of vector digested with the relevant restriction enzymes (e.g. pET21:χ1C1b digested with NotI and EcoRI or pET21:χ1C5 digested with NotI and NruI) was incubated with 4 μl of the annealed primers, 1 μl ligase buffer, 2 μl T4 DNA Ligase and the reaction made up to 10 μl with sterile water. These were incubated overnight in a beaker of ice, which was allowed to thaw over this time. 5 μl of the overnight ligation was then added to 50 μl of chemically competent E. coli SURE cells. This was incubated on ice for 1 hour, then heat shocked at 42° C. for 30 sec. 450 μl LB media was added to the cells and then the sample incubated for 30 min at 37° C. The mini-culture was then centrifuged for 5 min at 4000 rpm, the resulting pellet was re-suspended in 50 μl LB media, and then plated onto LB plates containing carbenicillin (100 μg/ml), tetracycline (10 μg/ml) and glucose (0.3% w/v). This was incubated overnight at 37° C. The resulting colonies were then screened to check if the linker variation was successful.

### Modifications to the General Strategy

The generation of constructs with rigid linkers using the restriction enzymes NotI and NruI to digest pET21:χ1C5 generated a large number of colonies on the negative control plates (no primer duplex in the ligation reaction) after transformation, hence it was difficult to screen for positive clones. This was rectified by dephosphorylating the digested vector; 15 μl of pET21:χ1C5 digested with NotI and NruI was mixed with 2 μl CIAP 10× buffer, 1 μl CIAP (Calf Intestinal Alkaline Phosphatase) (10 U/μl) and 2 μl sterile water. This was incubated at 37° C. for 1 hour and then at 80° C. for 30 min. The DNA was then cleaned from solution using a purification kit (e.g. Qiagen PCR Purification Kit). All the primers used to make the primer duplexes were phosphorylated using the method described above. The phosphorylated primer duplexes were then ligated into the dephosphorylated vector as described above.

### Cloning and Expression of χ1L1

χ1L1 consists of two domains of growth hormone GH followed by a single extracellular growth hormone domain, each of these domains are currently linked with a (Gly₄Ser)₄ linker (FIG. 8). The nucleotide sequence of χ1L1 is shown in FIG. 9 and the amino acid sequence is given in FIG. 10.

χ1L1 was constructed by ligating the hGH gene flanked by NheI and XhoI sites into χ1E2 (GHRa-GH-GHRb); this gave χ1K1. The GHR domain was then ligated into χ1K1 between EcoRI and HindIII sites to give χ1L1. A schematic diagram of this procedure is shown in FIG. 11.

The χ1L1 gene was checked by sequencing and was shown to be correct. Expression was carried out using a modified pET21(+) vector in E. coli BL21(DE3) CodonPlus RIPL cells. Protein expressed in LB media 4 hours after induction with 1 mM IPTG (final concentration) at an OD₆₀₀ of 0.5-0.6, was partially soluble and multiple Mw bands were observed in the western blot probed against GH (FIG. 12).

A C-terminal His-tagged version of χ1L1 was purified using a Co²⁺ column (FIG. 13). A number of contaminating proteins remained in the protein prep and the multiple Mw bands were still observed in the western blot. Preliminary bioassays of this protein preparation showed that it had significant agonistic activity (FIG. 14).

### Construction of Prolactin Tandems and Prolactin: Growth Hormone Tandems

Constructs of PRL and GH tandems are generated using standard PCR techniques followed by ligation and transformation of the prepared vector. The linker can be varied by ligating and transforming annealed oligonucleotides pairs into prepared vectors. Three example strategies are shown below for the construct of PRL and GH tandem.

### Strategy 1

### Generation of PRL-(G₄S)₄-PRL

1. PCR PRL between NcoI and NotI sites (forward primer=atatccatgggcTTGCCCATCTGTCC; reverse primer=atatatatatatgggcggccgccGCAGTTGTTGTTGTGG).
2. Digest PCR product with NcoI and NotI.
3. Digest recipient vector with NcoI and NotI→pET21(m)χ1C1b (i.e. GH-(G₄S)₄-GH).
4. Ligate PCR product into vector; to give pET21(m)PRL-(G₄S)₄-GH
5. PCR PRL between EcoRI and HindIII sites (forward primer=atatgaattcTTGCCCATCTGTCC; reverse primer=atataagcttGCAGTTGTTGTTGTGG).
6. Digest PCR product with EcoRI and HindIII.
7. Digest recipient vector with EcoRI and HindIII→pET21(m)PRL-(G₄S)₄-GH.
8. Ligate PCR product into vector; to give pET21(m)PRL-(G₄S)₄-PRL.

### Strategy 2

### Generation of PRL-A(EA₃K)₂A-GH

1 PCR PRL between NcoI and NotI sites (forward primer=atatccatgggcTTGCCCATCTGTCC; reverse primer=atatatatatatgggcggccgccGCAGTTGTTGTTGTGG).
2 Digest PCR product with NcoI and NotI.
3 Digest recipient vector with NcoI and NotI→pET21(m)T1aEAK2 (i.e. GH-A(EA₃K)₂A-GH).
4 Ligate PCR product into vector; to give PRL-A(EA₃K)₂A-GH.

### Strategy 3

### Generation of PRL-A(EA₃K)₄A-PRL

1. Anneal the primers for the generation of the A(EA₃K)₄A linker.
2. Digest recipient vector with NotI and EcoRI→pET21(m) PRL-(G₄S)₄-PRL (from example 1 above)
3. Ligate oligonucleotide dimer into vector; to give PRL-A(EA₃K)₄A-PRL

The above strategy is illustrated in FIG. 24.

### Example 1

### Semi-Rigid Tandems

E. coli BL21 (DE3) CodonPlus-RIPL cells were grown in 10 ml LB media supplemented with carbenicillin, tetracycline and choramphenicol. The cells were grown shaking at 37° C. The cultures were induced at an OD600 of 0.4-0.7 using IPTG to a final concentration of 1 mM. The cultures were grown for a further 4 hrs before harvesting. The cells were lysed using a combination of lysozyme, sodium deoxychloate and sonication. The soluble fraction was then isolated by centrifugation. A coomassie stained PAGE gels showed no obvious bands for tandem expression.

The soluble fraction was determined by ELISA, and 40 ng/well of tandem was loaded onto a 12% PAGE gel. The protein was transferred to PVDF membrane and the western blotted using rabbit anti-GH Ab (primary) and anti-rabbit-HRP Ab (secondary); see FIG. 16d. The bioactivity of a GH tandem comprising a semi-rigid linker is shown in FIG. 16e.

### Example 2

### Rigid Tandems

E. coli BL21(DE3)CodonPlus-RIPL cells were grown in 10 ml LB media supplemented with carbenicillin, tetracycline and choramphenicol. The cells were grown shaking at 37° C. The cultures were induced at an OD600 of 0.4-0.7 using IPTG to a final concentration of 1 mM. The cultures were grown for a further 4 hrs before harvesting.

The cells were lysed using a combination of lysozyme, sodium deoxychloate and sonication. The soluble fraction was then isolated by centrifugation. A coomassie stained PAGE gels showed no obvious bands for tandem expression

The soluble fraction was determined by ELISA, and 40 ng/well of tandem was loaded onto a 12% PAGE gel. The protein was transferred to PVDF membrane and the western blotted using rabbit anti-GH Ab (primary) and anti-rabbit-HRP Ab (secondary); see FIG. 17d. The bioactivity of a Gh tandem comprising a semi-rigid linker is shown in FIG. 17e.

### Example 3

### Purification of Tandems

Constructs T1cEAK2+3His and T1cEAK2+4His were short-listed for further study based on their initial supra-maximal activities in the bioassay. The expression plasmid was transformed into E. coli BL21(DE3) Codonplus RIPL cells and expression was carried out in 1 L batch cultures. Purification was performed on the soluble protein fraction using a combination of Ni-chelate immobilised metal-ion affinity chromatography (IMAC) and ion-exchange chromatography. IMAC was the first purification step, initially elution was achieved using a pH gradient (pH 8 to pH 3); however it was found that a lot of protein was being lost in the column washes. Therefore we returned to using an imidazole elution (0 to 0.5M imidazole), with modifications in the purification strategy we achieved >70% purity. An ion-exchange column (Resource Q) was then used to further purify the protein to >90% purity. This is illustrated in FIG. 18.

Quantification based on ELISA results: T1cEAK2+3His (RQ13/4) 215 μg/ml; T1cEAK2+3His (RQ14/4)=177 μg/ml

1 ml of each obtained hence the total yield was 392 μg. This was obtained from 2 litres of culture→yield per litre=˜200 μg

The activity of T1cEAK2+3-His reaches a higher fold induction than rhGH at the higher protein concentrations. A similar result is obtained when the tandem is tested on a molar basis, see FIG. 18B and FIG. 18C. A similar analysis was conducted with respect to T1cEAK2+4His. The purification and bioactivity is illustrated in FIGS. 18D, 18E and 18F.

Quantification based on ELISA results of T1cEAK2+4His (RQ13/4)=550 μg/ml. 1 ml of each obtained hence the total yield was 550 μg. This was obtained from 2 litres of culture→yield per litre=˜275 μg. The activity of T1cEAK2+4-His reaches a higher fold induction than rhGH at the higher protein concentrations. A similar result is obtained when the tandem is tested on a molar basis.

### Example 4

The concentration of χ1C3 was measured using the Bradford's Assay rhGH (@1 mg/ml) was measured in parallel to verify the veracity of the data obtained from the Bradford's Assay. χ1C3 was then used to directly replace the GH standards in the GH bioassay to give a tandem standard curve. Pure and impure tandem samples and rhGH were measured against the GH standard curve and the tandem standard curve, the protein concentration was then measured from each ELISA plate. The GH ELISA gives approximately two-thirds of the actual value of the tandems as measured by these ELISAs. This is illustrated in FIG. 19.

### Example 5

### Prolactin/GH Tandems

Tandems of prolactin and/or GH, with and without their respective antagonistic mutation, can be synthesized using PCR to introduce the appropriate restriction sites to either end of the gene to enable ligation into the tandem gene.

### Flexible Tandems

The tandem gene is constructed by linking two protein domains with a flexible linker based on the sequence (G₄S)ₙ; there are unique restriction sites at each end of the protein domains and the linker (FIG. 20).

Hence the two protein domains in the tandem can be varied by ligating in different domains. For example, the prolactin (PRL), prolactin 1-14 amino acid deleted G129R mutant (Δ1-14PRL .G129R), growth hormone (GH) and the growth hormone G120R antagonist mutant (GH.G120R) can be combined in the tandem gene in a variety of ways (Figure B). FIGS. 22 and 23 show the nucleotide sequences and protein sequences for these domains.

Standard PCR can be used to generate the genes for the desired protein domain to be flanked by the appropriate restriction endonuclease sites. Digestion of the PCR product and the recipient vector with these restriction endonucleases followed by ligation and transformation will generate a tandem with the desired protein domains. This process can be carried out on either protein domain or on the linker (FIG. 24), which would be replaced using an oligonucleotide dimer as already described.

### Semi-Rigid Tandems

The tandem gene is constructed by linking two protein domains with a helical linker based on the sequence A(EA₃K)ₙA; there are unique restriction sites at each end of the protein domains and the linker (FIG. 20).

Hence the two protein domains in the tandem can be varied by ligating in different domains. For example, the prolactin (PRL), prolactin 1-14 amino acid deleted G129R mutant (Δ1-14PRL .G129R), growth hormone (GH) and the growth hormone G120R antagonist mutant (GH.G120R) can be combined in the tandem gene in a variety of ways (FIG. 21). FIGS. 22 and 23 show the nucleotide sequences and protein sequences for these domains.

Standard PCR can be used to generate the genes for the desired protein domain to be flanked by the appropriate restriction endonuclease sites. Digestion of the PCR product and the recipient vector with these restriction endonucleases followed by ligation and transformation will generate a tandem with the desired protein domains. This process can be carried out on either protein domain or on the linker (FIG. 24), which would be replaced using an oligonucleotide dimer as already described.

### Rigid Tandems

The two domains of the tandem need to be linked directly through the C-terminus α-helix of domain A and the N terminal \acute{α}-helix of terminal B. Hence the genes for the proteins (FIGS. 22 and 23) at domain A and B need to be truncated so that the helical linker [A[EA₃K)ₙA] is joined directly to these helices.

Hence:—

| | Domain A | Domain B |
|---|---|---|
| Protein | truncation | truncation |
| GH | 184-191 | 1-6 |
| PRL | 191-199 | 1-14 |

The tandem gene is constructed by linking two protein domains with a helical linker based on the sequence A(EA₃K)ₙA; there are unique restriction sites at each end of the protein domains and the linker (FIG. 20). A unique NotI site has been engineered into the N-terminal end of the linker region and a unique NruI site has been engineered into the C-terminal end of GH (FIGS. 5 and 6); this enables the modification of the linker region in GH tandems.

The N-terminal linker sequence including the NotI site can be directly appended to a PRL gene in the domain A position allowing constructs based on the template PRL-linker-GH to be constructed (FIG. 25). However, a unique restriction site has to be introduced at the boundary between the linker and domain B in the cases where domain B is PRL (FIG. 25).

Hence the two protein domains in the tandem can be varied by ligating in different truncated domains. For example, the prolactin (PRL), prolactin 1-14 amino acid deleted G129R mutant (Δ1-14PRL .G129R), growth hormone (GH) and the growth hormone G120R antagonist mutant (GH.G120R) can be combined in the tandem gene in a variety of ways (FIG. 21). Depending on their position in domain A or domain B the protein domains will have to be truncated as described above.

Standard PCR can be used to generate the genes for the desired protein domain to be flanked by the appropriate restriction endonuclease sites. Digestion of the PCR product and the recipient vector with these restriction endonucleases followed by ligation and transformation will generate a tandem with the desired protein domains. This process can be carried out on either protein domain or on the linker and is similar to the methodology used for the flexible and semi-rigid linkers (FIG. 24).

1. A polypeptide comprising at least two cytokine binding domains capable of binding to a cytokine receptor, wherein the domains are linked by a peptide linker molecule that comprises an inflexible helical region.

2. A polypeptide according to claim 1 wherein said domains comprise 3, 4, 5, 6, 7, 8, 9, or 10 binding domains in a tandem array.

3. A polypeptide according to claim 1 wherein the polypeptide comprises more than 10 domains in a tandem array.

4. A polypeptide according to claim 1 wherein the inflexible helical region comprises at least one copy of the motif A(EAAAK)ₓA, or a functional variant thereof.

5. A polypeptide according to claim 1 wherein the linker molecule comprises at least one flexible non-helical region.

6. A polypeptide according to claim 5 wherein a flexible non-helical region is located at or near the amino-terminal end of the peptide linker molecule.

7. A polypeptide according to claim 5 wherein the flexible non-helical region is located at or near the carboxyl-terminal end of the peptide linker molecule.

8. A polypeptide according to claim 5 wherein the flexible non-helical region is located at or near the amino and the carboxyl-terminal end of the peptide linker molecule.

9. A polypeptide according to claim 5 wherein the flexible non-helical region is located adjacent to at least one of the binding domains.

10. A polypeptide according to claim 4 wherein the polypeptide comprises less than 10 copies of the EAAAK motif.

11. A polypeptide according to claim 4 wherein the polypeptide comprises less than 5 copies of the EAAAK motif.

12. A polypeptide according to claim 2 wherein said binding domains are linked by a linking molecule consisting of an inflexible helical linker.

13. A polypeptide according to claim 12 wherein said helical linker links the carboxyl terminus of one binding domain with the amino terminus of a second binding domain.

14. A polypeptide according to claim 13 wherein the helical linker is continuous between the C-terminal helix of the first binding domain and the N-terminal helix of the second binding domain, thus rigidly linking the two binding domains in a substantially fixed orientation

15. A polypeptide according to claim 1 wherein the binding domains of the polypeptide are the same or similar to each other.

16. A polypeptide according to claim 15 wherein the polypeptide comprises binding domains of cytokines selected from the group consisting of growth hormone; leptin; erythropoietin; prolactin; interleukins (IL) IL-2, IL-3, IL-4, IL-5, IL-6, IL-7, IL-9, IL-10, IL-11, the p35 subunit of IL-12, IL-13, IL-15; granulocyte colony stimulating factor (G-CSF); granulocyte macrophage colony stimulating factor (GM-CSF); ciliary neurotrophic factor (CNTF); cardiotrophin (CT-1); leukocyte inhibitory factor (LIF); oncostatin M (OSM); interferon, IFNa and IFNg; tumour necrosis factor (TNF)a and TNFb, and RANK ligand.

17. A polypeptide according to claim 16 wherein at least one of the domains comprises a growth hormone binding domain, or a growth hormone variant.

18. A polypeptide according to claim 17 wherein the polypeptide comprises at least two binding domains of growth hormone, or a growth hormone variant polypeptide.

19. A polypeptide according to claim 16 wherein said polypeptide comprises at least two binding domains of prolactin, or a prolactin variant.

20. A polypeptide according to claim 19 wherein said prolactin variant polypeptide comprises an amino acid sequence wherein said amino acid sequence is modified at position 129 of prolactin.

21. A polypeptide according to claim 20 wherein said modification is an amino acid substitution.

22. A polypeptide according to claim 21 wherein said substitution replaces a glycine amino acid residue with an arginine amino acid residue.

23. A polypeptide according to claim 19 wherein said polypeptide further comprises the deletion of between 9 and 14 amino terminal amino acid residues of prolactin.

24. A polypeptide according to claim 1 wherein the binding domains of the polypeptide are dissimilar to each other.

25. A polypeptide according to claim 24 wherein said polypeptide comprises a first binding domain that is a growth hormone binding domain and a second binding domain that is a prolactin binding domain.

26. A polypeptide according to claim 24 wherein said polypeptide consists of a growth hormone binding domain, the linker molecule, and a prolactin binding domain.

27. A polypeptide according to claim 24 wherein said polypeptide comprises a first binding domain that is a modified growth hormone binding domain and a second binding domain that is a modified prolactin binding domain.

28. A polypeptide according to claim 27 wherein said polypeptide consists of a modified growth hormone binding domain, the linker molecule, and a modified prolactin binding domain.

29. A polypeptide according to claim 27 wherein said modified growth hormone binding domain comprises an amino acid substitution at amino acid position glycine 120.

30. A polypeptide according to claim 29 wherein the modification is a replacement of glycine 120 by an amino acid selected from the group consisting of arginine, lysine, tryptophan, tyrosine, phenylalanine, and glutamic acid.

31. A polypeptide according to claim 30 wherein said modification is the replacement of glycine 120 with an arginine amino acid residue.

32. A polypeptide according to claim 27 wherein said modified prolactin binding domain comprises a modification of glycine 129.

33. A polypeptide according to claim 32 wherein said modification is the replacement of glycine 129 with an arginine amino acid residue.

34. A polypeptide according to claim 29 wherein said polypeptide further comprises the deletion of between 9 and 14 amino terminal amino acid residues of prolactin.

35. A polypeptide according to claim 1 wherein said polypeptide further comprises a ligand binding domain of a cytokine receptor.

36. A polypeptide according to claim 35 wherein said receptor is a growth hormone receptor.

37. A polypeptide according to claim 35 wherein said receptor is a prolactin receptor.

38. A nucleic acid molecule that encodes a polypeptide according to claim 1.

39. A nucleic acid according to claim 38 wherein said nucleic acid is a vector adapted for the expression of said polypeptide.

40. An isolated cell transformed or transfected with the vector according to claim 39.

41. An isolated cell according to claim 40 wherein said cell is a eukaryotic cell.

42. An isolated cell according to claim 40 wherein said cell is a prokaryotic cell.

43. A method of preparing a polypeptide comprising at least two cytokine binding domains capable of binding to a cytokine receptor, wherein the domains are linked by a peptide linker molecule that comprises an inflexible helical region, said method comprising the steps of
i) growing a cell according to claim 40 in conditions conducive to the production of a polypeptide encoded by the nucleic acid of the vector with which said cell has been transformed or transfected; and
ii) isolating the polypeptide from the cell, or its growth environment.

44. A polypeptide comprising a first cytokine binding domain linked to a second cytokine binding domain wherein said polypeptide further comprises an extracellular domain of a cytokine receptor.

45. A polypeptide according to claim 44 wherein said first and second binding domains are linked by a flexible linker molecule.

46. A polypeptide according to claim 44 wherein said first and second binding domains are linked by a peptide linker molecule that comprises an inflexible helical region.

47. A polypeptide according to claim 44 wherein said first and second binding domains are linked by a peptide linker molecule comprising an inflexible helical region and a flexible, non-helical region.

48. A polypeptide according to claim 44 wherein said cytokine binding domain is growth hormone, or a growth hormone variant thereof, and said extracellular domain is a growth hormone extracellular domain.

49. A nucleic acid molecule that encodes a polypeptide according to claim 44.

50. A nucleic acid molecule according to claim 49 wherein said nucleic acid is a vector adapted for the expression of said polypeptide.

51-52. (canceled)

53. A composition comprising the polypeptide of claim 1 or a nucleic acid encoding said polypeptide, and at least one pharmaceutically acceptable carrier or adjuvant.

54. An isolated cell transformed or transfected with a vector according to claim 50.

55. An isolated cell according to claim 54 wherein said cell is a eukaryotic cell.

56. An isolated cell according to claim 54 wherein said cell is a prokaryotic cell.
